(19) United States
(12) Patent Application Publication
(10) Pub. No.: US 2013/0052198 A1
(43) Pub. Date: Feb. 28, 2013

(54) ANTI-INFLAMMATORY FACTORS

(71) Applicants: John Miles Milwid, Cambridge, MA (US); Biju Parekkadan, Cambridge, MA (US); Martin Leon Yarmush, Newton, MA (US); Matthew LI, Cambridge, MA (US)

(72) Inventors: John Miles Milwid, Cambridge, MA (US); Biju Parekkadan, Cambridge, MA (US); Martin Leon Yarmush, Newton, MA (US); Matthew LI, Cambridge, MA (US)

(73) Assignees: THE GENERAL HOSPITAL CORPORATION, Boston, MA (US); MASSACHUSETTS INSTITUTE OF TECHNOLOGY, Cambridge, MA (US)

(21) Appl. No.: 13/638,368

(22) PCT Filed: Mar. 29, 2011
(86) PCT No.: PCT/US11/30310

§ 371 Date: Oct. 26, 2012

Related U.S. Application Data
(60) Provisional application No. 61/318,604, filed on Mar. 29, 2010.

(51) Int. Cl. A61K 38/45; C12Q 1/37; A61K 39/395; A61P 29/00; A61K 38/17; A61K 38/22; G01N 33/566; C40B 30/04

(52) U.S. Cl. 424/137.1; 435/7.92; 435/23; 506/9; 424/94.5; 514/1.1; 514/20.9; 514/9.7; 424/172.1; 424/158.1

(57) ABSTRACT

The present invention provides particular anti-inflammatory factors, compositions containing them, methods of making, identifying, and/or characterizing them, and methods of using them. In some embodiments, provided factors are expressed by human bone marrow stromal cells (MSC). In some embodiments, provided factors are characterized by an ability, when contacted with mammalian leukocytes in culture, to alter production of at least one inflammatory or anti-inflammatory agent by the mammalian leukocytes. In some embodiments, provided factors include GALNT1 polypeptides, LGALS3BP polypeptides, MFAP5 polypeptides, PENK polypeptides and/or HAPLN1 polypeptides. In some embodiments, provided factors are useful in the inhibition of inflammatory agents, in the promotion of anti-inflammatory agents, and/or for the treatment of subjects suffering from or susceptible to a disease, disorder or condition characterized by inflammation.

### RELATED APPLICATIONS

The present application is a national phase application under 35 USC §371 of PCT International Application No. PCT/US2011/030310 (published PCT Application No. WO/2011/126833 A3), filed Mar. 29, 2011, which claims the benefit of U.S. Provisional Patent Application Ser. No. 61/318,604, filed Mar. 29, 2010; the entire contents of which are incorporated herein by reference.

### GOVERNMENT SUPPORT

The United States Government has provided grant support utilized in the development of the present invention. In particular, the National Human Genome Research Institute (NHGRI) grant number T32 HG002295 and National Institutes of Health (NIH) grant numbers K01DK087770, and R01DK43371 have supported development of this invention. The United States Government has certain rights in the invention.

### BACKGROUND

Marrow stromal cells (MSCs) are multipotent adult progenitor cells that are being clinically explored as a new therapeutic for treating a variety of immune-mediated diseases. First heralded as a regenerative therapy for skeletal tissue repair, MSCs have recently been shown to modulate endogenous tissue and immune cells.

MSCs are currently being explored for use in humans because of their potent ability to treat many devastating diseases in animals including acute kidney injury, myocardial infarction, type I diabetes, graft vs. host disease, systemic lupus erythrematosus, multiple sclerosis, pulmonary fibrosis and stroke. While the primary mechanisms of action are yet to be fully elucidated, studies indicate that MSCs can act on several levels of endogenous repair to bring about resolution of disease. MSCs have been shown to protect cells from injury and directly promote tissue repair (Ortiz, Gambelli et al. 2003; Rojas, Xu et al. 2005). When administered to treat animals undergoing acute renal failure, MSCs prevent apoptosis and encourage proliferation of renal tubule epithelial cells in a differentiation-independent manner (Togel, Hu et al. 2005; Togel, Weiss et al. 2007). When injected into the myocardium after infarction, MSCs can differentiate into cardiomyocytes and reduce the incidence of scar formation (Shake, Gruber et al. 2002; Amado, Saliaris et al. 2005; Miyahara, Nagaya et al. 2006). When administered to prevent the onset of type I diabetes mellitus, MSCs protect β-islets from autoimmune attack and promote temporary restoration of glucose regulation when administered after onset of the disease, suggesting protection and repair of damaged islet tissues (Fiorina, Jurewicz et al. 2009).

In addition to promoting tissue repair directly, MSCs have also been shown to modulate the immune system and attenuate tissue damage caused by excessive inflammation. Initial indications as to the immunomodulatory aspects of MSCs were first observed in the context of MSC transplantation studies in animals and humans. Unexpectedly, MSCs seemed to exhibit an unusual ability to evade the immune system. Initial clinical trials showed that autologous and allogeneic MSCs could be transplanted without immune rejection (Lazarus, Haynesworth et al. 1995; Horwitz, Prockop et al. 1999). Further preclinical studies presented similar findings: human MSCs can engraft and persist in many tissues in prenatal and adult sheep with no apparent rejection (Liechty, MacKenzie et al. 2000); MSC injection in baboons can prolong the life of a transplanted skin graft and suppress T cell proliferation in a dose-dependent manner (Bartholomew, Sturgeon et al. 2002); and injected MSCs can suppress the immune response in mice, and allow for the expansion of tumor cells (Djouad, Plence et al. 2003). The immunosuppressive ability was first exploited clinically in the treatment of an 8-year old boy with severe, acute graft-versus host disease (GvHD), who was refractory to steroid immunosuppression (Le Blanc, Rasmusson et al. 2004). The patient was successfully treated by MSC transplantation. In recent years, this immunosuppression has been found to be an active process and the mechanisms underlying MSC immunomodulation operate at different levels of the innate and adaptive immune system.

Many studies suggest that MSCs can promote the conversion from a TH1 (cell-mediated) to TH2 (humoral) immune response (Aggarwal and Pittenger 2005). In vitro co-culture experiments have been used to exemplify the effects of MSCs on individual populations of immune cells that favor this conversion at the cellular and molecular level. With respect to adaptive immunity, the majority of in vitro studies have shown that MSCs can directly inhibit CD3+, CD4+ T cell proliferation and secretion of TH1 lymphokines, such as IL-2 and IFN-γ, induced by mixed lymphocyte reactions (MLR), mitogens and TCR or costimulatory receptor engagement. T cells in the presence of MSCs appear to be anergized by the lack of a second danger signal by MSCs, which do not express the co-stimulatory molecules CD80, CD86 and CD40 (Klyushnenkova, Mosca et al. 2005), however this has yet to be definitively proven. Several investigations have also shown a direct suppressive effect of MSCs on cytotoxic CD8+ T cells. MSCs prevented cytolysis of target cells by alloantigen-specific CD8+ T cells when present during the priming of cytotoxic cells (Rasmusson, Uhlin et al. 2007). Some investigators attribute the inhibition of cytotoxicity by MSCs to an intrinsic “veto” function or the generation of suppressor CD8+ cells after coculture (Potian, Aviv et al. 2003), although conflicting data exist. Nonetheless, other reports have also observed generation of CD4+ CD25+ T cells, a cell surface marker expression pattern of both newly activated CD4+ lymphocytes and regulatory T cell (Aggarwal and Pittenger 2005; Prevosto, Zancolli et al. 2007). Whether MSCs directly influence B cells in vivo, some in vitro evidence suggests that MSCs can suppress B cell proliferation (Augello, Tasso et al. 2005; Corcione, Benvenuto et al. 2006). Some reports have shown that MSCs can stimulate antibody secretion and induce polyclonal differentiation and expansion of healthy human B cells (Rasmusson, Le Blanc et al. 2007; Traggiai, Volpi et al. 2008), consistent with the supportive role of stromal cells in B lymphopoiesis. In addition, these same supportive mechanisms may advance the progression of B cell-mediated disease such as multiple myeloma and systemic lupus erythematosus (Arnulf, Lecourt et al. 2007; Traggiai, Volpi et al. 2008). However, it is likely that suppression of T cells by MSCs ultimately contribute to decreased B cell activity in vivo (Gerdoni, Gallo et al. 2007).

Within an inflamed tissue environment, MSCs are capable of influencing many aspects of the cytotoxic responses to injury and disease (Uccelli, Moretta et al. 2008). MSCs can attenuate natural cytotoxic responses of neutrophils by dampening respiratory burst and inhibiting spontaneous apoptosis in vitro via secretion of IL-6 (Raffaghello, Bianchi et al. 2008). MSCs also possess the ability to suppress proliferation of natural killer (NK) cells (Poggi, Prevosto et al. 2005; Sotiropoulou, Perez et al. 2006; Spaggiari, Capobianco et al. 2008), and attenuate their cytotoxic activity by downregulating the expression of NKp30 and NKG2D, surface receptors involved in NK cell activation (Spaggiari, Capobianco et al. 2006). This is accomplished even while cytokine-activated NK cells are capable of killing MSCs in vitro, suggesting a possible mechanism for MSC rejection in vivo (Spaggiari, Capobianco et al. 2008). MSCs can also revert macrophages to adopt an anti-inflammatory phenotype in the context of sepsis by secreting prostaglandin E2 and conveying a contact-dependent signal to promote IL-10 secretion (Németh, Leelahavanichkul et al. 2008).

Dendritic cells (DCs) are the major link between innate and adaptive immunity due to their ability to present antigens with high efficiency to lymphocytes. In coculture with MSCs, monocytes failed to differentiate into DCs when cultured in lineage-specifying growth conditions (Beyth, Borovsky et al. 2005; Jiang, Zhang et al. 2005). In addition, MSCs inhibited the maturation of DCs to present appropriate antigens and costimulation to T cells through CD1a, CD40, CD80, CD86, and HLA-DR (Zhang, Ge et al. 2004; Beyth, Borovsky et al. 2005). After coculture with MSCs, DCs were ineffective in their ability to activate lymphocytes by suppressing TNF-α and IFN-γ expression and upregulating IL-10 in DC-CD4+ MLRs (Jiang, Zhang et al. 2005). This interaction was found to be γ-secretase dependent, indicating the role of the Notch pathway in MSC-DC interactions (Li, Paczesny et al. 2008). Ultimately, MSCs may drive, or “license”, DCs to a suppressor phenotype which can further attenuate T cell-mediated immunity.

Preclinical studies of the mechanism of action suggest that the therapeutic effects afforded by MSC transplantation are short-lived and related to secreted interactions between MSCs and host cells. Efforts have been made to identify secreted factors that are predominantly responsible for the therapeutic activity of MSCs in immune-mediated diseases using biased approaches (Pittenger 2009). To-date, no single factor has been identified that induces the same potent and reproducible reversal of the inflammatory state of an animal or human as MSC transplantation.

### SUMMARY

Among other things, the present invention provides the identification of individual factors that are secreted by human MSCs using an objective screening approach. The present invention demonstrates that these individual factors cause an anti-inflammatory effect when exogenously delivered. The present invention provides isolated preparations of such factors, as well as compositions containing them, methods of using them, systems for assessing their presence in a sample, etc.

Thus, for example, the present invention provides particular factors, compositions containing them, methods of making, identifying, and/or characterizing them, and methods of using them.

In some embodiments, provided factors are polypeptides. In some embodiments, provided factors are produced and/or secreted by bone marrow stromal cells.

In some embodiments, provided factors polypeptides have immunomodulatory properties. In some embodiments, provided factors are characterized by an ability, when contacted with mammalian leukocytes in culture, to alter production of at least one pro-inflammatory or anti-inflammatory agent by the mammalian leukocytes. In some such embodiments, production is altered at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is altered at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments, production is increased. In some such embodiments, production is inhibited. In some such embodiments the at least one agent is a pro-inflammatory agent. In some such embodiments, the at least one agent is an anti-inflammatory agent. In some such embodiments, the at least one pro-inflammatory agent is selected from the group consisting of interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and combinations thereof. In some such embodiments, the at least one anti-inflammatory agent is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor, and combinations thereof; in some such embodiments, at least one anti-inflammatory agent is or includes IL-10; in some such embodiments, at least one pro-inflammatory agent is or includes IFN-γ.

In some embodiments, provided polypeptides are characterized in that, when one or more is/are administered to colitic mice, one or more features of their colitis is/are attenuated.

In some embodiments, provided polypeptides exert their effects when present at concentrations comparable to those at which such factors are naturally found in human serum. In some embodiments, provided polypeptides exert their effects when present at a concentration comparable to that at which a particular reference factor is naturally found in human serum.

In some embodiments, the present invention provides GALNT1 polypeptides, compositions containing them, nucleic acids encoding them (and/or complements of such nucleic acids), antibodies that recognize them, and/or methods or making or using such.

In some embodiments, the present invention provides LGALS3BP polypeptides, compositions containing them, nucleic acids encoding them (and/or complements of such nucleic acids), antibodies that recognize them, and/or methods or making or using such.

In some embodiments, the present invention provides MFAP5 polypeptides, compositions containing them, nucleic acids encoding them (and/or complements of such nucleic acids), antibodies that recognize them, and/or methods or making or using such.

In some embodiments, the present invention provides PENK polypeptides, compositions containing them, nucleic acids encoding them (and/or complements of such nucleic acids), antibodies that recognize them, and/or methods or making or using such.

In some embodiments, the present invention provides HAPLN1 polypeptides, compositions containing them, nucleic acids encoding them (and/or complements of such nucleic acids), antibodies that recognize them, and/or methods or making or using such.

In some embodiments, compositions provided herein are useful in the inhibition of inflammatory agents and/or in the promotion of anti-inflammatory agents. In some embodiments, such inflammatory agents are selected from the group consisting of interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18 and interleukin-8, and combinations thereof. In some embodiments, such anti-inflammatory agents are selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof. In some embodiments, such anti-inflammatory agents are or include IL-10. In some embodiments, such pro-inflammatory agents are or include IFN-γ.

In some embodiments, compositions provided herein are useful in medicine.

In some embodiments, compositions provided herein are useful in the treatment of subjects suffering from or susceptible to a disease, disorder or condition. In some such embodiments, the disease, disorder or condition is characterized by inflammation. In some such embodiments, the disease, disorder, or condition is selected from the group consisting of those listed in Table 3, and combinations thereof. In some such embodiments, the disease, disorder, or condition is selected from the group consisting of rheumatoid arthritis, type I and type II diabetes, ulcerative colitis, Crohn's disease, celiac disease, multiple sclerosis, myocardial infarction, neoplasm, chronic infectious disease, systemic lupus erythematosus, acute kidney injury, sepsis, multiple organ dysfunction syndrome, acute liver failure, chronic liver failure, chronic kidney failure, pancreatitis, Grave's disease, and combinations thereof.

The invention described herein provides a new approach to the treatment of inflammatory diseases, disorders and conditions through the use of purified factors, which Applicants have shown promote induction of anti-inflammatory agents and/or suppress the induction of pro-inflammatory agents.

Other features and advantages of the invention will be apparent from the following detailed description thereof, and from the claims.

### DEFINITIONS

Affinity: As is known in the art, “affinity” is a measure of the tightness with which a particular entity binds to its partner. Affinities can be measured in different ways.

Amino acid: As used herein, the term “amino acid,” in its broadest sense, refers to any compound and/or substance that can be incorporated into a peptide chain. In some embodiments, an amino acid has the general structure H2N—C(H)(R)—COOH. In some embodiments, an amino acid is a naturally-occurring amino acid. In some embodiments, an amino acid is a synthetic or unnatural amino acid; in some embodiments, an amino acid is a d-amino acid; in some embodiments, an amino acid is an 1-amino acid. Amino acids, including carboxy- and/or amino-terminal amino acids, can be modified by methylation, amidation, acetylation, and/or substitution with other chemical groups that can change the peptide's circulating half-life without adversely affecting its activity. An amino acid may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Antibody: As used herein, the term “antibody” refers to any immunoglobulin, whether natural or wholly or partially synthetically produced. All derivatives thereof which maintain specific binding ability are also included in the term. The term also covers any protein having a binding domain that is homologous or largely homologous to an immunoglobulin binding domain. Such proteins may be derived from natural sources, or partly or wholly synthetically produced. In some embodiments, an antibody is monoclonal. In some embodiments, an antibody is polyclonal. In some embodiments, an antibody is a single chain antibody. Those of ordinary skill in the art will appreciate that antibodies may be provided in any of a variety of forms including, for example, humanized, partially humanized, chimeric, chimeric humanized, etc. An antibody may be a member of any immunoglobulin class, including any of the human classes: IgG, IgM, IgA, IgD, and IgE, and of any immunoglobulin subclass (e.g., IgG1, IgG2, IgG3, or IgG4). Typically, an intact antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. A heavy chain constant region is comprised of three or four domains, CH1, CH2, CH3, and CH4, depending on the isotype. Each light chain is comprised of a light chain variable region (abbreviated herein as VL) and a light chain constant region. A light chain constant region is comprised of one domain, CL. VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. Variable regions of heavy and light chains contain a binding domain that interacts with an antigen. Constant regions of antibodies may mediate binding of antibodies to host tissues or factors, including various cells of the immune system (e.g., effector cells) and the first component (Clq) of the classical complement system. As used herein, the terms “antibody fragment” (i.e., “antigen-binding portion”) or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids, at least about 100 amino acids, at least about 150 amino acids, or at least about 200 amino acids.

Antigen binding portion: The term “antigen binding portion” of an antibody, as used herein, refers to one or more fragments of an intact antibody that retain the ability to specifically bind to a given antigen. Antigen binding functions of an antibody can typically be performed by fragments of an intact antibody. Examples of binding fragments encompassed within the term “antigen binding portion” of an antibody include an Fab fragment, a monovalent fragment comprising VL, VH, CL and CH1 domains; an F(ab)2 fragment, a bivalent fragment comprising two Fab fragments (generally one from a heavy chain and one from a light chain) linked by a disulfide bridge at the hinge region; an Fd fragment consisting of the VH and CH1 domains; an Fv fragment comprising the VL and VH domains of a single arm of an antibody and a single domain antibody (dAb) fragment (Ward et al., 1989 Nature, 341:544-546), that contains a VH domain; and an isolated complementarity determining region (CDR). Although the two domains of the Fv fragment, VL and VH, are usually encoded by separate genes, they can be joined (e.g., using recombinant technology and/or by including a linker polypeptide) as a single protein chain in which the VL and VH regions pair to form monovalent molecules (known as single chain Fv (scFv); see, e.g., Bird et al., 1988 Science, 242:423-426; and Huston et al., 1988 Proc. Natl. Acad. Sci., 85:5879-5883). Such single chain antibodies include one or more “antigen binding portions” of an antibody. Antibody fragments may be obtained or produced using conventional techniques known to those of skill in the art; if desired, fragments can be screened for binding in the same manner as are intact antibodies. Alternatively or additionally, antigen binding portions can be incorporated into single domain antibodies, maxibodies, minibodies, intrabodies, diabodies, triabodies, tetrabodies, v-NAR and bis-scFv (see, e.g., Hollinger and Hudson, 2005 Nature Biotechnology, 23(9):1126-1136). Similarly, antigen binding portions can alternatively or additionally be incorporated into single chain molecules comprising a pair of tandem Fv segments (VH—CH1-VH—CH1) which, together with complementary light chain polypeptides, form a pair of antigen binding regions (Zapata et al., 1995 Protein Eng., 8(10):1057-1062; and U.S. Pat. No. 5,641,870).

Anti-inflammatory agent: The term “anti-inflammatory agent”, as used herein, refers to a factor made by a leukocyte or other cell to counteract the effects of inflammatory cytokines and other agents that mimic the actions of inflammatory cytokines. In some embodiments, an anti-inflammatory agent is a cytokine. In some embodiments, and anti-inflammatory agent is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof. In some embodiments, an agent that mimics one or more biological activities of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof may be considered to be an anti-inflammatory agent as described herein.

Binding: It will be understood that the term “binding”, as used herein, typically refers to a non-covalent association between or among entities. In some embodiments, binding is addressed with respect to particular moieties of a targeting agent. It will be appreciated by those of ordinary skill in the art that such binding may be assessed in any of a variety of contexts. In some embodiments, an agent “specifically binds”, meaning that it discriminates between its intended target and other materials present within the sample with which it is contacted.

Characteristic sequence: A “characteristic sequence” is a sequence that is found in all members of a family of polypeptides or nucleic acids, but not in polypeptides or nucleic acids not in the family, and therefore can be used by those of ordinary skill in the art to define members of the family. In some embodiments, a characteristic sequence element in a polypeptide comprises a stretch of contiguous amino acids, typically 5 amino acids, e.g., at least 5-500, at least 5-250, at least 5-100, at least 5-75, at least 5-50, at least 5-25, at least 5-15, or at least 5-10 amino acids, that shows at least about 80%, at least 85%, at least 90%, at least 95%, at least 96%, at least 97%, at least 98%, at least 99%, or 100% identity with other polypeptides in the family or class. In some embodiments, a characteristic sequence element participates in or confers function on a polypeptide.

Comparable to: The term “comparable to” is sometimes used herein to refer specifically to an amount of a factor used in a particular context as compared with an amount of such a factor naturally found in human serum. In some embodiments, an amount is considered to be “comparable to” a reference amount if it is within an order of magnitude (i.e., ten times higher or lower than) of the reference amount. In some embodiments, an amount is considered to be “comparable to” a reference amount if it is within 9, 8, 7, 6, 5, 4, 3, 2, 1.9, 1.8, 1.7, 1.6, 1.5, 1.4, 1.3, 1.2, or 1.1 fold (i.e., times higher or lower than) the reference amount.

Corresponding to: As used herein, the term “corresponding to” is used to designate the position/identity of an amino acid residue in a polypeptide of interest, as compared with a reference polypeptide. In general, an amino acid residue in a polypeptide of interest is a residue that is found in a corresponding sequence context and/or performs a corresponding role to its cognate residue in the reference polypeptide. Those of ordinary skill in the art are well familiar with strategies and technologies for performing sequence comparisons and can readily identify corresponding amino acids. For example, as is well known in the art, two or more nucleotide or amino acid sequences can be aligned using standard bioinformatic tools, including programs such as BLAST, ClustalX, Sequencher, etc. Even though the two or more sequences may not match exactly and/or do not have the same length, an alignment of the sequences can still be performed and, if desirable, a “consensus” sequence generated. Indeed, programs and algorithms used for alignments typically tolerate definable levels of differences, including insertions, deletions, inversions, polymorphisms, point mutations, etc. Such alignments can aid in the determination of which positions in one sequence correspond to which positions in other sequences.

Dosing Regimen: A “dosing regimen”, as that term is used herein, refers to a set of unit doses (typically more than one) that are administered individually separated by periods of time. In some embodiments, a dosing regimen is a therapeutic regimen.

Factor: the term “factor” as used herein, refers to an agent of any chemical class or composition that has the recited characteristic or property. For example, a factor may be or include small molecules, biological molecules (e.g., polypeptides, lipids, carbohydrates, nucleic acids, etc., including for example, glycoproteins, glycolipids, proteoglycans, lipoproteins, etc.), metals, vitamins, etc. A factor may be comprised of a single chemical entity, or may comprise a collection of chemical entities associated with one another (e.g., in a complex).

GALNT1 polypeptide: The term “GALNT1 polypeptide”, as used herein, refers to a polypeptide that shares certain structural and/or functional characteristics with UDP-N-acetyl-α-D-galactosamine:polypeptide N-acetylgalactosaminyltransferase 1 (GalNac-T1; GALNT1) as described herein. Reference GALNT1 polypeptides have sequences presented in Table 2. In some embodiments, an GALNT1 polypeptide is characterized in that it shows at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more overall sequence identity with a sequence presented in Table 2. In some embodiments, an GALNT1 polypeptide is characterized in that it includes an amino acid sequence element found in a sequence selected from the group consisting of GenBank Accession Numbers: AAH90583.1, AAH47746.1, AAH38440.1, AAH90962.1, AAH56215.1, BAI47186.1, EAX01360.1, EAX01359.1, CAD44535.1, EDL76133.1, EDK96994.1, EDK96993.1; NCBI Reference Sequence Numbers: NP_{—}065207.2, NP_{—}001083410.1, NP_{—}001153876.1, NP_{—}038842.3, NP_{—}077349.1, NP_{—}803485.1, NP_{—}001025547.1, NP_{—}001006381.1; and combinations thereof. In some embodiments, a GALNT1 polypeptide is characterized in that it includes an amino acid sequence element found in a sequence selected from the group consisting of any of the sequences presented in Table 2 and SEQ ID NO: 24-438.

In some embodiments, a GALNT1 polypeptide has an amino acid sequence identical to that of a polypeptide that is naturally produced by marrow stromal cells. In some embodiments, a GALNT1 polypeptide is characterized by an ability, when contacted with mammalian leukocytes in culture, to increase production of at least one anti-inflammatory agent (and/or to decrease production of at least one pro-inflammatory agent) by the mammalian leukocytes. In some such embodiments, production is increased (or decreased) at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is increased (or decreased) at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments the at least one anti-inflammatory agent is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof; in some such embodiments, the at least one anti-inflammatory agent is or includes IL-10. In some embodiments, a GALNT1 polypeptide is characterized in that, when it is administered to colitic mice, one or more features of their colitis is/are attenuated. The sequence corresponding to GenBank Accession Number AAH47746.1 (SEQ ID NO: 224), which is a GALNT1 polypeptide, is typically expressed in mammalian cells at a level within the range of 0.21 fg*cell⁻¹*hour⁻¹.

Gene: The term “gene” is used herein according to its art-understood meaning to refer to a sequence of nucleotides that is expressed in a cell. In some embodiments, a gene may encode one or more polypeptides, including for example, polypeptides that are related to one another as splice variants. In some embodiments, a gene includes one or more introns; in some embodiments a gene does not include any introns. In some embodiments, a gene is referred to by reference to the name of a polypeptide that it encodes.

Human antibody: As used herein, the term “human antibody”, includes antibodies having variable regions in which both the framework and CDR regions are derived from sequences of human origin. In certain embodiments, if an antibody contains a constant region, the constant region also is derived from such human sequences, e.g., human germline sequences, or mutated versions of human germline sequences. Human antibodies may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

In combination: The phrase “in combination”, as used herein, refers to two or more agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents are present (e.g., at relevant levels) in the subject's body, they are considered to be administered “in combination”.

Inflammatory agent: The term “inflammatory agent” (or “pro-inflammatory agent”), as used herein, refers to a factor made by a leukocyte or other cell in response to an inflammatory stimulus. In some embodiments, an inflammatory agent is a cytokine. In some embodiments, an inflammatory agent is selected from the group consisting of interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18 and interleukin-8, and combinations thereof. In some embodiments, an agent that mimics one or more biological activities of interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18 and interleukin-8, and/or combinations thereof may be considered to be an inflammatory cytokine. By “inflammatory cytokine” is meant a protein made by a leukocyte or other cell in response to an inflammatory stimulus.

Isolated: The term “isolated,” is used herein, to describe an agent or entity that has either (i) been separated from at least some of the components with which it was associated when initially produced (whether in nature or in an experimental setting); and/or (ii) produced by the hand of man. Isolated agents or entities may be separated from at least about 10%, at least about 20%, at least about 30%, at least about 40%, at least about 50%, at least about 60%, at least about 70%, at least about 80%, at least about 90%, or more of the other components with which they were initially associated. An isolated entity may be partially or completely pure. A partially pure agent or entity is substantially free of other materials (e.g., is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more pure).

LGALS3BP polypeptide: The term “LGALS3BP polypeptide”, as used herein, refers to a polypeptide that shares certain structural and/or functional characteristics with lectin, galactoside-binding, soluble, 3 binding protein (LGALS3BP) as described herein. Reference LGALS3BP polypeptides have sequences presented in Table 2. In some embodiments, an LGALS3BP polypeptide is characterized in that it shows at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more overall sequence identity with a sequence presented in Table 2. In some embodiments, an LGALS3BP polypeptide is characterized in that it includes an amino acid sequence element found in a sequence selected from the group consisting of GenBank Accession Numbers: CAM23109.1, AAA36193.1, AAI14269.1, AAH81724.1, AAH90658.1, AAH15761.1, AAH02998.1, AAH02403.1; BAI45468.1, EAW89546.1, EAW89545.1, EAW89544.1, EAW89543.1, CAM23110.1, ABM86350.1, AAI05368.1, EDM06756.1, EDM06755.1, EDL34662.1, EDL34661.1, ABM83150.1; NCBI Reference Sequence Numbers: NP_{—}005558.1, NP_{—}620796.1, NP_{—}035280.1, NP_{—}001035130.1; and combinations thereof. In some embodiments, an LGALS3BP polypeptide is characterized in that it includes an amino acid sequence element found in a sequence selected from the group consisting of any of the sequences presented in Table 2, FIG. 4, and SEQ ID NO: 329-353. In some embodiments, an LGALS3BP polypeptide has an amino acid sequence identical to that of a polypeptide produced by marrow stromal cells. In some embodiments, an LGALS3BP polypeptide is characterized by an ability, when contacted with mammalian leukocytes in culture, to increase production of at least one anti-inflammatory agent (and/or to decrease production of at least one pro-inflammatory agent) by the mammalian leukocytes. In some such embodiments, production is increased (or decreased) at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is increased (or decreased) at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments the at least one anti-inflammatory agent is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof; in some such embodiments, the at least one anti-inflammatory agent is or includes IL-10. In some embodiments, an LGALS3BP polypeptide is characterized in that, when it is administered to colitic mice, one or more features of their colitis is attenuated. The sequence corresponding to GenBank Accession Number AAH02998 (SEQ ID NO: 339), which is an LGALS3BP, is typically expressed in mammalian cells at a level within the range of 4.2 fg*cell⁻¹*hour⁻¹.

Marrow stromal cell: The term “marrow stromal cell”, as used herein, is meant to be synonymous with mesenchymal stem cell and mesenchymal stromal cell. As is known in the art, such cells are found in (and can be isolated from) such tissues as, but not limited to, bone marrow, adipose tissue, placental tissue, amniotic fluid, synovial fluid or joints, lymph nodes, thymus, spleen, testes, skin. Alternatively or additionally, marrow stromal cells for use in accordance with the present invention can be derived from another stem cell such as an embryonic stem cell. In some embodiments, a marrow stromal cell is a cell that exhibits an immunophenotype including, but not limited to the markers CD11−, CD14−, CD18−, CD31−, CD34−, CD40−, CD45−, CD56−, CD80−, CD86−, MHCII−, CD29+, CD44+, CD71+, CD73+, CD90+, CD105+, CD106+, CD120a+, CD124, CD166+, Stro-1+, ICAM-1+, MHCI+.

Marrow stromal cell factors: The term “marrow stromal cell factors”, as used herein, refers in general to factors that are naturally produced by marrow stromal cells. Such factors may be of any chemical class, including, for example, polypeptides, lipids, carbohydrates, nucleic acids, etc. (including, for example, glycoproteins, glycolipids, proteoglycans, lipoproteins, etc). Specifically provided marrow stromal cell factors include gene products produced by stromal cells that, when isolated from the cells, modulate inflammatory activity. In some embodiments, provided marrow stromal cell factors include products of GALNT1, LGALS3BP, MFAP5, PENK and/or HAPLN1 genes. For example, in some embodiments, provided marrow stromal cell factors include GALNT1 polypeptides, nucleic acids encoding them (and/or complements of such nucleic acids); LGALS3BP polypeptides, nucleic acids encoding them (and/or complements of such nucleic acids); MFAP5 polypeptides, nucleic acids encoding them (and/or complements of such nucleic acids); PENK polypeptides, nucleic acids encoding them (and/or complements of such nucleic acids); and combinations thereof). It will be appreciated by those of ordinary skill in the art that a marrow stromal cell factor as described herein retains its identity as a marrow stromal cell factor regardless of its mode of production. In some embodiments, marrow stromal cell factors described herein are in fact produced by or in marrow stromal cells. In some embodiments, marrow stromal cell factors described herein are produced in alternative systems (e.g., in a recombinant system such as a eukaryotic or prokaryotic cell that has been engineered by the hand of man to produce the marrow stromal cell factor and/or in a cell-free or synthetic system).

MFAP5 polypeptide: The term “MFAP5 polypeptide”, as used herein, refers to a polypeptide that shares certain structural and/or functional characteristics with microfibrillar-associated protein 5 (MFAP5) as described herein. Reference MFAP5 polypeptides have sequences presented in Table 1. In some embodiments, an MFAP5 polypeptide is characterized in that it shows at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more overall sequence identity with a sequence presented in Table 1. In some embodiments, an MFAP5 polypeptide is characterized in that it includes an amino acid sequence element found in a sequence selected from the group consisting of GenBank Accession Numbers: AAH05901.1, AAA96752.1, AAD53950.1, EAW88614.1, EAW88613.1, AAH25131.1, ACE87011.1, AAI02770.1, EDK99687.1, EDK99686.1, EDK99685.1; and NCBI Reference Sequence Numbers: NP_{—}056591.1, NP_{—}003471.1, NP_{—}776811.1, NP_{—}001102114.1; and combinations thereof. In some embodiments, an MFAP5 polypeptide is characterized in that it includes an amino acid sequence element found in a sequence selected from the group consisting of any of the sequences presented in Table 2 and SEQ ID NO: 130-144. In some embodiments, an MFAP5 polypeptide has an amino acid sequence identical to that of a polypeptide that is naturally produced by marrow stromal cells. In some embodiments, an MFAP5 polypeptide is characterized by an ability, when contacted with mammalian leukocytes in culture, to increase production of at least one anti-inflammatory agent (and/or decrease production of at least one pro-inflammatory agent) by the mammalian leukocytes. In some such embodiments, production is increased (or decreased) at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is increased (or decreased) at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments the at least one anti-inflammatory agent is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof; in some such embodiments, the at least one anti-inflammatory agent is or includes IL-10. In some embodiments, an MFAP5 polypeptide is characterized in that, when it is administered to colitic mice, one or more features of their colitis is attenuated. The sequence corresponding to NCBI Reference Sequence Number NP_{—}003471.1 (SEQ ID NO: 136), which is a MFAP5 polypeptide, is typically expressed in mammalian cells at a level within the range of 209 fg*cell⁻¹*hour⁻¹.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleic acid residues. In some embodiments, a “nucleic acid” is or comprises RNA and/or DNA. In some embodiments, a “nucleic acid” is partially or wholly single stranded; in some embodiments, a “nucleic acid” is partially or wholly double stranded. In some embodiments, a “nucleic acid” described herein may include one or more nucleic acid analogs. In some embodiments, a nucleic acid may include “peptide nucleic acids,” which are known in the art and have peptide bonds instead of phosphodiester bonds in the backbone. The term “nucleotide sequence encoding an amino acid sequence” refers to the set of nucleotide sequences that are degenerate versions of each other and/or encode the same amino acid sequence. In some embodiments, a nucleic acid may include introns. Nucleic acids can be prepared according to any available technique, including, for example, isolation from natural sources, recombinant expression, chemical synthesis, etc. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid analog: The term “nucleic acid analog” is used herein to refer to a nucleic acid having a non-natural feature. In some embodiments, a nucleic acid analog has other than a phosphodiester backbone. In some embodiments, a nucleic acid analog has a non-natural base or sugar, etc. In some embodiments, analogs have modified bases or sugars and/or backbone modifications, etc. as compared with a reference natural nucleic acid.

Nucleic acid segment: The term “nucleic acid segment” is used herein to refer to a nucleic acid sequence that is a portion of a longer nucleic acid sequence. In some embodiments, a nucleic acid segment comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65, at least 70, at least 75, at least 80, at least 86, at least 90, at least 95, at least 100, or more residues.

PENK polypeptide: The term “PENK polypeptide”, as used herein, refers to a polypeptide that shares certain structural and/or functional characteristics with proenkephalin (PENK; also called preproenkephalin and/or proenkephalin A) as described herein. Reference PENK polypeptides have sequences presented in Table 2. In some embodiments, a PENK polypeptide is characterized in that it shows at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more overall sequence identity with a sequence presented in Table 1. In some embodiments, a PENK polypeptide is characterized in that it includes an amino acid sequence element found in a sequence selected from the group consisting of GenBank Accession Numbers: CAG46627.1, CAG46607.1, AAH90311.1, AAI07707.1, AAH83563.1, ACI66659.1, AAH32505.1, AAV84279.1, AAB59409.1, ABM84953.1, ABM81791.1, ABM81797.1, AAH78348.2, AAI11280.1; NCBI Reference Sequence Numbers: NP_{—}001129162.1, NP_{—}006202.1, NP_{—}001002927.1, NP_{—}001088343.1, NP_{—}001015744.1, NP_{—}001166888.1, NP_{—}878303.1, NP_{—}058835.1, NP_{—}776566.1, NP_{—}001135308.1; and combinations thereof. In some embodiments, a PENK polypeptide is characterized in that it includes an amino acid sequence element found in a sequence selected from the group consisting of any of the sequences presented in Table 1, Table 2, and SEQ ID NO: 194-217. In some embodiments, a PENK polypeptide has an amino acid sequence identical to that of a polypeptide that is naturally produced by marrow stromal cells. In some embodiments, a PENK polypeptide is characterized by an ability, when contacted with mammalian leukocytes in culture, to increase production of at least one anti-inflammatory agent (and/or to decrease production of at least one pro-inflammatory agent) by the mammalian leukocytes. In some such embodiments, production is increased (or decreased) at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is increased (or decreased) at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments the at least one anti-inflammatory agent is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof; in some such embodiments, the at least one anti-inflammatory agent is or includes IL-10. In some embodiments, a PENK polypeptide is characterized in that, when it is administered to colitic mice, one or more features of their colitis is/are attenuated. The sequence corresponding to GenBank Accession Number AAH32505 (SEQ ID NO: 209), which is a PENK polypeptide, is typically expressed in mammalian cells at a level within the range of 4.2 fg*cell⁻¹*hour⁻¹.

HAPLN1 polypeptide: The term “HAPLN1 polypeptide”, as used herein, refers to a polypeptide that shares certain structural and/or functional characteristics with Hyaluronan and proteoglycan link protein 1 (HAPLN1) as described herein. Reference HAPLN1 polypeptides have sequences presented in Table 2. In some embodiments, an HAPLN1 polypeptide is characterized in that it shows at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more overall sequence identity with a sequence presented in Table 2. In some embodiments, an HAPLN1 polypeptide is characterized in that it includes an amino acid sequence element found in a sequence selected from the group consisting of GenBank Accession Number: AAH57808.1, AAI51456.1, BAD52342.1, AAH66853.1, BAI47315.1, EAW95914.1, EAW95913.1, EAW95912.1, EDM09988.1, EDL00984.1, AAI28741.1; NCBI Reference Sequence Number: NP_{—}038528.3, NP_{—}001875.1, NP_{—}001007791.1, NP_{—}001075973.1; and combinations thereof. In some embodiments, an HAPLN1 polypeptide is characterized in that it includes an amino acid sequence element found in a sequence selected from the group consisting of any of the sequences presented in Table 2, FIG. 7, and SEQ ID NO: 41-55. In some embodiments, an HAPLN1 polypeptide has an amino acid sequence identical to that of a polypeptide that is naturally produced by marrow stromal cells. In some embodiments, an HAPLN1 polypeptide is characterized by an ability, when contacted with mammalian leukocytes in culture, to decrease production of at least one pro-inflammatory agent (and/or to increase production of at least one anti-inflammatory agent) by the mammalian leukocytes. In some such embodiments, production is decreased (or increased) at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is decreased (or increased) at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments the at least one pro-inflammatory agent is selected from the group consisting of interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18 and interleukin-8, and combinations thereof; in some such embodiments, at least one pro-inflammatory agent is or includes IFN-γ. In some embodiments, an HAPLN1 polypeptide is characterized in that, when it is administered to subjects with diseases with inflammatory diseases, one or more features of their inflammatory disease is attenuated. The sequence corresponding to GenBank Accession Number: AAH57808.1 (SEQ ID NO: 41), which is an HAPLN1 polypeptide, is naturally typically expressed in mammalian cells at a level within the range of 4.2 fg*cell⁻¹*hour⁻¹.

Polypeptide: The term “polypeptide”, as used herein, generally has its art-recognized meaning of a polymer of at least three amino acids. In some embodiments, a polypeptide comprises natural amino acids. In some embodiments, a polypeptide comprises one or more amino acid analogs (i.e., entities that can be incorporated into a polypeptide chain via a peptide bond). In some embodiments, one or more residues in a polypeptide is not a natural amino acid and/or contains a modification (e.g., an attached glycan group or other polymer group, etc) as compared with a reference natural amino acid. The term “polypeptide” is also used herein to refer to amino acid polymers that share a degree of sequence identity and/or biological functionality. For example, in some embodiments, a “polypeptide” has an amino acid sequence exactly as recited herein. In some embodiments, a “polypeptide” has an amino acid sequence that is a fragment of a sequence that is recited herein (or in a reference or database specifically mentioned herein); in some such embodiments, the fragment shows some or all of the biological activities of the polypeptide having the complete recited sequence. In some such embodiments, a fragment comprises at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300 amino acids, or more. In some embodiments, a “polypeptide” shares at least about 30-40% overall sequence identity, often greater than about 50%, greater than about 60%, greater than about 70%, or greater than about 80%, and/or includes at least one region of much higher identity, often greater than 90% or even greater than 95%, greater than 96%, greater than 97%, greater than 98%, or greater than 99% in one or more highly conserved regions, usually encompassing at least 3-4 and often up to 20 or more amino acids, with another polypeptide (i.e., a reference polypeptide, for example, of the same class). Other regions of similarity and/or identity can be determined by those of ordinary skill in the art by analysis of the sequences of various polypeptides. In some embodiments, two or more polypeptides are considered to be of the same class because they show such overall sequence identity and/or share one or more characteristic sequence elements. In some embodiments, two or more polypeptides are considered to be of the same class because they share one or more biological activities and one or more common structural features (e.g., a given level of overall sequence identity and/or one or more characteristic sequence elements). In general, polypeptides described herein may be produced by any available means. For example, in some embodiments, a polypeptide may be isolated from a natural source. In some embodiments, a polypeptide may be produce recombinantly (i.e., in or from a host cell engineered by the hand of man to produce the polypeptide). In some embodiments, a polypeptide may be produced in a cell-free system. In some embodiments, a polypeptide may be synthesized.

Pure: As used herein, an agent or entity is “pure” if it is substantially free of other components. For example, in some embodiments, a preparation that contains more than about 90% of a particular agent or entity is typically considered to be a pure preparation. In some embodiments, an agent or entity is at least 90%, at least 91%, at least 92%, at least 93%, at least 94%, at least 95%, at least 96%, at least 97%, at least 98%, or at least 99% pure.

Sample: As used herein, a “test sample” refers to a biological sample obtained from a subject of interest. In some embodiments, a “test sample” comprises a biological fluid (e.g., blood, joint fluid, mucous, saliva, semen, synovial fluid, tears, urine, etc). In some embodiments, a “test sample” comprises one or more cells. In some embodiments, a “test sample” comprises tissue. In some embodiments, a test sample is a sample that is processed (e.g., by one or more of filtration, distillation, extraction, concentration, inactivation of interfering components, addition of reagents, and the like) from a raw sample obtained directly from the subject. In some embodiments, a processed sample is partially or completely purified.

Specificity: As is known in the art, “specificity” is a measure of the ability of a particular entity to distinguish first binding partner from one or more other available potential binding partners. In some embodiments, an entity having specificity has at least 50%, at least 100%, at least 2-fold, at least 3-fold, at least 4-fold, at least 5-fold, at least 10-fold, at least 20-fold, at least 50-fold, at least 100-fold, at least 200-fold, at least 500-fold, or at least 1000-fold higher affinity for a first binding partner than for a second binding partner.

Subject: As used herein, the term “subject” or “patient” refers to an organism to which a composition described herein may be administered, e.g., for experimental, diagnostic, prophylactic, and/or therapeutic purposes, and/or from which a sample may be obtained. Typical subjects include animals (e.g., mammals such as mice, rats, rabbits, non-human primates, and humans; insects; worms; etc.). In many embodiments, a subject is a mammal. In many embodiments, a subject is a human.

Suffering from: An individual who is “suffering from” a disease, disorder, and/or condition (e.g., disease, disorder or condition characterized by inflammation) has been diagnosed with and/or displays one or more symptoms of a disease, disorder, and/or condition.

Susceptible to: An individual who is “susceptible to” a disease, disorder, and/or condition (e.g., disease, disorder or condition characterized by inflammation) has not been diagnosed with a disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the phrase “therapeutic agent” refers to any agent that elicits a desired biological or pharmacological effect. In some embodiments, a therapeutic agent elicits a desired biological or pharmacological effect when administered in a therapeutic regimen.

Therapeutic regimen: A “therapeutic regimen” typically comprises a collection of individual doses of a therapeutic agent, delivered according to a determined schedule and via a designated route or routes. In many embodiments, a therapeutic regimen is one whose use correlates with achievement of a particular therapeutic effect in an organism (e.g., an animal or human).

Therapeutically effective amount: The term “therapeutically effective amount” of an agent or combination of agents is intended to refer to an amount of agent(s) which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). A therapeutically effective amount is commonly administered in a dosing regimen that may comprise multiple unit doses. In some embodiments of the present invention, a composition may be considered to contain a therapeutically effective amount of an agent(s) if it contains an amount appropriate for administration as a unit dose in the context of a therapeutic regimen. For any particular pharmaceutical agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific pharmaceutical agent employed; the duration of the treatment; and like factors as is well known in the medical arts.

Treatment: As used herein, the term “treatment” refers to any method used to partially or completely alleviate, ameliorate, relieve, inhibit, prevent, delay onset of, reduce severity of, reduce incidence of, and/or yield prophylaxis of one or more symptoms or aspects of a disease, disorder, or condition. In some embodiments, treatment can involve administration of one or more doses before, during, and/or after onset of symptoms.

Unit dose: The term “unit dose”, as used herein, refers to a discrete administration of a pharmaceutical agent, typically in the context of a dosing regimen.

Variant: As used herein, the term “variant” is a relative term that describes the relationship between a particular polypeptide (e.g., a myostatin antagonist polypeptide having a sequence similar to that of myostatin) of interest and a reference polypeptide (e.g., in many embodiments, a wild type polypeptide) to which its sequence is being compared. A polypeptide of interest is considered to be a “variant” of a reference polypeptide if the polypeptide of interest has an amino acid sequence that is identical to that of the reference but for a small number of sequence alterations (e.g., insertions, substitutions, and/or deletions) at particular positions. Typically, fewer than 20%, fewer than 15%, fewer than 10%, fewer than 9%, fewer than 8%, fewer than 7%, fewer than 6%, fewer than 5%, fewer than 4%, fewer than 3%, fewer than 2%, fewer than 1%, or fewer of the residues in the variant are altered as compared with the parent. In some embodiments, a variant has 10, 9, 8, 7, 6, 5, 4, 3, 2, or 1 altered residues as compared with a parent. Often, a variant has a very small number (e.g., fewer than 5, fewer than 4, fewer than 3, fewer than 2, or fewer than 1) number of altered functional residues (i.e., residues that participate in a particular biological activity). In some embodiments, a variant not more than 5, not more than 4, not more than 3, not more than 2, or not more than 1 additions or deletions as compared with the reference polypeptide; in many embodiments, a variant has no additions or deletions (although it may have one or more substitutions) as compared with the reference polypeptide. In many embodiments, variants that contain additions and/or deletions contain additions and/or deletions of fewer than about 25, fewer than about 20, fewer than about 19, fewer than about 18, fewer than about 17, fewer than about 16, fewer than about 15, fewer than about 14, fewer than about 13, fewer than about 10, fewer than about 9, fewer than about 8, fewer than about 7, fewer than about 6, residues, and commonly are fewer than about 5, fewer than about 4, fewer than about 3, fewer than about 2, or fewer than about 1 residue. In some embodiments, the parent polypeptide is one found in nature.

Vector: As used herein, “vector” refers to a nucleic acid molecule capable of transporting another nucleic acid to which it has been linked. In some embodiment, vectors are capable of extra-chromosomal replication and/or expression of nucleic acids to which they are linked in a host cell such as a eukaryotic or prokaryotic cell. Vectors capable of directing the expression of operatively linked genes are referred to herein as “expression vectors.”

Wild type: As is understood in the art, the phrase “wild type” generally refers to a normal form of a protein or nucleic acid, as is found in nature. For example, wild type polypeptides found in nature (e.g., from mammalian sources, such as human, pig, cow, etc.) include those presented in Table 2. Those of ordinary skill will understand how to identify wild type polypeptides and will understand the scope of the term as used herein.

### DESCRIPTION OF THE DRAWING

The Drawing of the present disclosure is comprised of the following Figures:

FIG. 1: MSC conditioned medium causes peripheral blood mononuclear cells to secrete IL-10 in response to LPS, providing the basis for differential gene expression analysis to identify genes responsible for the increased activity. (a) Generalized schematic of EPS methodology. Protein products are derived from various cell types in the form of conditioned media and screened for activity in an in vitro potency assay. Based on the activity of the conditioned media from the cells, hierarchical comparative gene expression profiling is performed to select for genes uniquely upregulated in the cell type with the highest activity in the potency assay. Recombinant protein products of the enriched gene list are then screened in a potency assay and the candidates with the highest activity are assessed for activity in vivo. (b) In vitro potency assay. This assay entails incubating primary human peripheral blood mononuclear cells (PBMCs) in the presence of protein products (e.g., conditioned medium from a cell) for 16 hours, followed by stimulation of the PBMCs with LPS for five hours, and measurement of IL-10 secretion into the supernatant via ELISA. (c) Time course of IL-10 expression from PBMCs when incubated with either bone marrow stromal cell conditioned medium (BMSC-CM) or unconditioned medium (DMEM) in the potency assay. (d) Comparison of potency assay activity of conditioned medium from normal human dermal fibroblasts (FB-CM), BMSCs (BMSC-CM) and BMSCs preincubated with LPS prior to conditioning (BMSC_{LPS}-CM). * p<0.001 compared to FB-CM, ** p<0.001 compared to BMSC-CM. (e) Schematic of the differential gene expression comparison strategy to select for genes uniquely expressed at high levels in LPS stimulated MSCs (MSC_{LPS}) compared to MSCs and fibroblasts (FBs). Analysis was then conducted on the genes identified to select for genes that were most likely to be responsible for secreted proteins. MSC_{LPS}, LPS stimulated MSC; FB, fibroblast. (f) Gene expression profiling revealed 22 genes responsible for secreted proteins that were upregulated by BMSCs stimulated with LPS compared to BMSCs and FBs.

FIG. 2: Characterization of IL-10 assay and optimization of BMSC preconditioning. (a) Kinetics of IL-10 secretion by PBMCs incubated with or without BMSC-CM and stimulated with LPS. (b) Dose response of the potency assay to increasing concentration of BMSC-CM. 1×CM was either diluted or concentrated further to generate the different concentrations. * p<0.001 compared to DMEM. (c) Effect of proteinase K on the activity of BMSC-CM in the potency assay. (d) Effects of preconditioning on the activity of BMSC-CM in the potency assay.

FIG. 3: Liquid chromatography of BMSC-CM Chromatography was used to fractionate the BMSC-CM into 0.5 mL fractions and then the fractions were evaluated for IL-10 activity in the potency assay. (a) Fractions generated by size exclusion chromatography. (b) Fractions generated by anion exchange chromatography.

FIG. 4: Screen of recombinant proteins that reveals four factors capable of significantly upregulating IL-10 in PBMCs. Recombinant proteins from 22 genes were acquired and screened for their ability to cause an upregulation of IL-10 in peripheral blood mononuclear cells stimulated with LPS. (a) Of the recombinant proteins screened, four exhibited a greater than 3-fold increase of IL-10 secretion over baseline at concentrations in the range of 5 μg/mL, GALNT1, LGALS3BP, MFAP5 and PENK. The normalized secretion described here is in reference to baseline IL-10 secretion in PBMCs never exposed to recombinant protein. (b) The presence of LGALS3BP, and MFAP5 were confirmed in the MSC conditioned medium via ELISA and western blotting. * p<0.001 compared to FB-CM. (c) Partial list of proteins contained in 10×FB-CM, BMSC-CM, and BMSC_{LPS}-CM detected by proteomic mass spectrometry. Mass spectrometry of the MSC conditioned medium confirmed the presence of six proteins identified in the gene screen as secreted factors of MSCs. The sequences presented in panel (c) are as follows:

(SEQ ID NO: 1)
PAQGVVTTLENVSPPR
(SEQ ID NO: 2)
EINLAPDSSSVVVSGLMVATK
(SEQ ID NO: 3)
IYTSPTWSAFVTDSSWSAR
(SEQ ID NO: 4)
TLQALEFHTVPFQLLAR
(SEQ ID NO: 5)
YDALEVFAGSGTSGQR
(SEQ ID NO: 6)
GESGYVASEGFPNYLPPNK
(SEQ ID NO: 7)
VYTAQNPSAQALGLGK
(SEQ ID NO: 8)
EFDDDTYDNDIALLQLK
(SEQ ID NO: 9)
QFQADFTSLSDQEPLHVAQALQK
(SEQ ID NO: 10)
VFQQVAQASK
(SEQ ID NO: 11)
NLPSDSQDLGQHGLEEDFM*L
(SEQ ID NO: 12)
GPMFELLPGESNK

FIG. 5: Three proteins expressed by MSCs and LPS-stimulated MSCs exhibit IL-10 activity that is comparable to MSC-CM. From the list of genes upregulated in LPS-stimulated MSCs compared to MSCs and fibroblasts, 18 proteins were chosen for the initial screen and six positive hits identified. The three highlighted in this figure induce the highest production of IL-10 in the IL-10 assay. (a) Compared to 1× and 10×MSC-CM activity normalized by baseline expression of IL-10 by PBMCs incubated with basal medium, three factors exhibited superior increased production of IL-10. Proteins were serially diluted and then subjected to the same PBMC assay conditions as performed on MSC-CM. (b) All three proteins identified exhibited a unique window of activity as identified by order of magnitude serial dilution. LGALS3PB, galactin-3-binding protein; MFAP5, microfibrillar-associated protein 5; GALNT1, UDP-N-acetyl-α-D-galactosamine:polypeptide N-acetylgalactosaminyltransferase 1.

FIG. 6: Administered MFAP5 and LGALS3BP protect mice from developing TNBS colitis. Mice were presensitized via the skin with 1% TNBS one week prior to intra-rectal administration of 3 mg of TNBS per mouse in a mixture of 50% EtOH and 50% sterile water to induce colitis. Mice were treated with 3 μg each of MFAP5, LGALS3BP and GALNT1 twice over a 24 hour period, once in conjunction with the TNBS administration and once 24 hours later. Mice were then sacrificed at day 2 and tissue collected for analysis. As expected, mice treated with vehicle (saline) developed extensive inflammation of the colon with loss of crypts and frank necrosis of the intestinal epithelium. Mice treated with GALNT1 were not protected from development of colitis as evidenced by similar histopathology. In contrast, mice treated with MFAP5 or LGALS3BP were protected from developing extensive colonic inflammation. Colons from MFAP5 and LGALS3BP treated animals exhibited only mild edema and minor foci of inflamed tissue.

FIG. 7. (A) MSC-CM was found to cause a significant decrease in IFN-γ production of the cultured leukocytes once stimulated with LPS. As also seen in this Figure, IFN-γ production of the leukocytes incubated with Fb-CM was similar in amount to using the same volume of RPMI medium as a control. In contrast, LPS-stimulated MSC-CM reduced IFN-γ production even lower than MSCs that had not received prior stimulation with LPS. These results indicated that MSCs secrete specific factors that decrease IFN-γ production from leukocytes in a manner that fit the prerequisites for our differential gene expression analysis. From the analysis performed in Example 2, HAPLN1 was identified and observed to independently reduce IFN-γ production from leukocytes when used in a purified, recombinant form. (B) These results indicated a dose-dependency of IFN-γ production over several orders of magnitude of diluted HAPLN1 in RPMI 1640. FIG. 7 also presents the identifying sequence of HAPLN1 found in MSC-CM by size separation liquid chromatography followed by mass spectrometry (bottom panel) (GGSDSDASLVITDLTLEDYGR, SEQ ID NO: 13).

FIG. 8: In vivo hit screen and survival study. (a) Schematic of the in vivo LPS assay. Proteins were administered intraperitoneal (IP) at the concentration that elicited the strongest effect in vitro, followed by IP administration of LPS in conjunction with a second dose of the proteins 16 hours later. Two days after the combined LPS and second protein dose, the mice were sacrificed and assessed for changes in serum cytokines and tissue histology. (b) Serum IL-10 levels of BALB/cJ mice subjected to the in vivo LPS assay. * p<0.001 compared to saline. (c) Serum TNF-α levels of BALB/cJ mice subjected to the in vivo LPS assay. * p<0.001 compared to saline, ** p<0.05 compared to BMSC-CM. (d) Representative micrographs of lung tissue from mice subjected to the in vivo LPS assay stained with hematoxylin and eosin. (e) Survival of mice subjected to a lethal dose of LPS i.p. (350 μg) concurrently with i.p. saline vehicle (bold dark blue line), 5 μg Anti-TNF-α antibody (thin dark blue line), 4 μg PENK (bold light blue line) or 4 μg MFAP5 (thin light blue line). Scale bar=200 μm.

FIG. 9. Listing of SEQ ID numbers for exemplary sequences of TFPI2, HAPLN1, PCOLCE2, FNDC1, LIF, MFAP5, INHBA, SRGN, CRISPLD1, ADAMTSL1, PENK, CDCP1, GALNT1, CRLF1, CFH, FN1, SERPINE1, HBEGF, LGALS3BP, BMP2, IGFBP1, and APOL1. This figure also presents exemplary entrez gene numbers encoding for provided polypeptides.

### DESCRIPTION OF CERTAIN EMBODIMENTS

### Bone Marrow Stromal Cell Factors

Therapeutic benefits observed when MSCs are transplanted can be completely recapitulated, and in some cases improved upon, by administration of MSC-conditioned supernatants. This finding is consistent with the observation that suppression in most MSC-immune cell co-culture studies can be reproduced in the absence of cell-cell contact and in a dose-dependent manner.

MSC-conditioned supernatants have no anti-proliferative effect on T cells, yet are capable of suppressing the stimulation of B cells (Augello, Tasso et al. 2005). This finding suggests that MSCs can dynamically react to their immunological environment in the context of T cells, while also secreting immunomodulatory agents in their quiescent, undifferentiated state in the context of B cell development. Also, there is an approximate 1-2 order of magnitude difference between the number of MSCs needed to suppress T cell activity compared to B cell activity (Le Blanc 2003; Corcione, Benvenuto et al. 2006). These studies hint at the interesting dynamics and dosing of MSC-derived factors to consider when evaluating MSC therapeutic applications.

Which soluble mediators are involved in MSC therapy (reviewed in (van Poll, Parekkadan et al. 2008)), has remained a topic of significant debate, although a clear distinction can be made that some molecules are considered naturally secreted by MSCs and others are inducible. Many candidates such as hepatocyte growth factor (HGF), transforming growth factor β₁ (TGF-β₁), or the metabolic byproduct of indoleamine 2,3-dioxygenase (IDO) activity (Klyushnenkova, Mosca et al. 1998; Tse, Beyer et al. 2000; Di Nicola, Carlo-Stella et al. 2002; Le Blanc, Tammik et al. 2003; Meisel, Zibert et al. 2004; Aggarwal and Pittenger 2005) are basally secreted by these cells. However, stimulation of MSCs by toll-like receptor ligands or inflammatory cytokines causes an alteration of the MSC secretome and a different set of chemical species (Block, Ohkouchi et al. 2009; Yagi, Parekkadan et al. 2009). For example, LPS found in serum leads to the rapid upregulation of prostaglandin E₂ (PGE₂), likely through an immediate early gene response related to NF-κB. Recently, a direct correlation between the upregulation of an anti-inflammatory protein, TSG-6, upon engraftment of MSCs in the lungs and the recovery of myocardial function after infarction was demonstrated (Lee, Pulin et al. 2009). Nevertheless, despite extensive investigation into the most potent factors secreted by MSCs, none have been shown to elicit the same therapeutic response as transplanted MSCs. Many factors have been proposed to compose the majority of MSC therapeutic activity including HLA-G, IL-6, IL-1Rag, IL-10, PGE2, TGFβ, Gal-1, and HGF; however, none of these factors have been shown to possess sufficient activity to account for the therapeutic potency of MSCs (Pittenger 2009). Indeed, prior to the present disclosure, all previous approaches have been biased, and never has it been demonstrated or indeed hypothesized that MSCs secrete the polypeptides of GALNT1, LGALS3BP, MFAP5, HAPLN1, and/or PENK. Furthermore, the present disclosure encompasses the recognition and represents the first demonstration that these polypeptides, provided in isolated form (e.g., without MSCs and/or having been produced recombinantly or synthetically or otherwise than by MSCs) cause a change in cytokine secretion of immune cells when administered exogenously to a subject and/or have anti-inflammatory activity.

The present invention demonstrates, among other things, that certain factors that are naturally produced by marrow stromal cells that modulate cytokine production. The present invention identifies such factors, isolates and characterizes them, and demonstrates their activities. Those of ordinary skill in the art, reviewing the present specification, including the data and guidance provided herein, will readily appreciate that the present invention provides, for example, GALNT1 polypeptides, compositions containing them, nucleic acids encoding them (and/or complements of such nucleic acids), and/or methods or making or using such; LGALS3BP polypeptides, compositions containing them, nucleic acids encoding them (and/or complements of such nucleic acids), and/or methods or making or using such; MFAP5 polypeptides, compositions containing them, nucleic acids encoding them (and/or complements of such nucleic acids), and/or methods or making or using such; PENK polypeptides, compositions containing them, nucleic acids encoding them (and/or complements of such nucleic acids), and/or methods or making or using such; HAPLN1 polypeptides, compositions containing them, nucleic acids encoding them (and/or complements of such nucleic acids); and combinations thereof.

The present invention therefore provides particular factors (e.g., GALNT1 polypeptides, nucleic acids encoding them (and/or complements of such nucleic acids); LGALS3BP polypeptides, nucleic acids encoding them (and/or complements of such nucleic acids); MFAP5 polypeptides, nucleic acids encoding them (and/or complements of such nucleic acids); PENK polypeptides, nucleic acids encoding them (and/or complements of such nucleic acids); HAPLN1 polypeptides, nucleic acids encoding them (and/or complements of such nucleic acids); and combinations thereof).

GALNT1

Polypeptide N-acetylgalactosaminyltransferase 1 (GALNT1; a.k.a. GalNAc-T1) is a mucin-type O-linked glycosylation enzyme that is primarily active in the Golgi (White, Bennett et al. 1995). Unlike other members of the UDP-N-acetyl-alpha-D-galactosamine:polypeptide N-acetylgalactosaminyltransferase family of enzymes, GALNT1 is a secreted molecule with enzymatic activity to promote extracellular glycosylation. GALNT1 is a type II membrane protein, possessing a cytoplasmic N-terminal domain, a transmembrane domain, a stem domain and a catalytic domain (Imberty, Piller et al. 1997). The catalytic domain is thought to contain a Rossman-type nucleotide binding domain in addition to a lectin-binding domain (Breton, Oriol et al. 1996; Imberty, Piller et al. 1997). The enzymatic activity of GALNT1 is dependent on a region of conserved cysteines that are required for mucin-type O-linking of glycans (Tenno, Toba et al. 2002). Its enzymatic activity is significantly reduced in its secreted form, suggesting the possibility of alternative extracellular functions (Zhu, Allende et al. 1998). Of note, there are no instances describing any association of GALNT1 with the regulation or stimulation of inflammatory or anti-inflammatory mediators. The present invention demonstrates a causal role of exogenously delivered GALNT1 in the treatment of immune diseases by an anti-inflammatory mechanism. One specific example is presented in colitis. The present invention therefore provides GALNT1 polypeptides, and various related compositions and methods.

LGALS3BP Polypeptides

Soluble galectin 3 binding protein (LGALS3BP; a.k.a. 90K, Mac2 binding protein, CyCAP) is a highly glycosylated secreted protein which binds galectin-1, galectin-3 and galectin-7 (Rosenberg, Cherayil et al. 1991). It is synthesized and secreted by different cell types, including hematopoietic cells and glandular or mucosal epithelia (Koths, Taylor et al. 1993; Ullrich, Sures et al. 1994) and is present in the serum and other biologic fluids of normal subjects in the μg/ml range (D'Ostilio, Sabatino et al. 1996). The present disclosure establishes, among other things, that a human mesenchymal cell secretes LGALS3BP.

LGALS3BP is one member of the scavenger receptor cysteine-rich domain superfamily that includes CD5, CD6, M130, complement factor 1, WC1, and other proteins that are structurally reminiscent of immunoglobulins (Resnick, Pearson et al. 1994). Under non-dissociative conditions and at neutral pH, LGALS3BP exists as on oligomer of multiple units that aggregate to form a collective mass that ranges of 1000-1500 kDa (Sasaki, Brakebusch et al. 1998). One individual 97 kDa subunit may be catalytically cleaved into 70 and 27 kDa fragments. It is glycosylated at a number of sites which may be critical to its bioactivity as well as its ability to be solubilized in different mediums such as serum or breast milk.

Based on the role of galectins in cell-cell and cell-matrix interactions, LGALS3BP has been associated with diseases in mammals that involve these interactions (Sasaki, Brakebusch et al. 1998). LGALS3BP is elevated in patients with cancer and viral infections, where in many instances its serum level has been found to be independent and inversely correlated of survival (Marchetti, Tinari et al. 2002). Clinical manifestation of pouchitis is inversely correlated with galectin-3 expression in the pouches' subepithelial lamina propria macrophages (Brazowski, Dotan et al. 2009).

A knockout mouse for LGALS3BP exists (Trahey and Weissman 1999) and has been found to spontaneously develop colonic mucosal hyperplasia and exaggerated tumorigenesis after treatment with carcinogen azoxymethane (Torlakovic, Keeler et al. 2009). Its role in inflammatory diseases has been controversial. Some have reported that it may be a potentiator of an immune response (Ullrich, Sures et al. 1994).

The present disclosure demonstrates a causal role of exogenously delivered LGALS3BP in the treatment of immune diseases by an anti-inflammatory mechanism. One specific example is presented in colitis. The present invention therefore provides LGALS3BP polypeptides, and various related compositions and methods.

MFAP5

Microfibrillar-associated protein 5 (MFAP5; a.k.a. microfibril-associated glycoprotein-2, MAGP-2) is an ECM glycoprotein localized to microfibrils and associated with elastin networks. It is a highly hydrophilic molecule consisting of two distinct domains: a cysteine-free acidic N-terminal half, and a cysteine-rich basic C-terminal half. It has been found to have significant homology (57%) with MFAP2 (MAPG-1) (Gibson, Hatzinikolas et al. 1996), in particular with respect to the cysteine rich region. A series of 7 cysteines near to the center of both molecules precisely align when their sequences are compared, and the cysteine separation distances are highly conserved. MFAP5 binds fibrillin-1 and -2 at the C-terminus, as well as to other proteins containing EGF-like repeats (Penner, Rock et al. 2002). It contains an RGD integrin-binding motif and has been shown to bind integrin (Gibson, Leavesley et al. 1999). MFAP5 has been shown to interact with the Notch receptor pathway (Miyamoto, Lau et al. 2006). Conflicting accounts describe this interaction as inhibitory (Albig, Becenti et al. 2008), and activating (Miyamoto, Lau et al. 2006), so the mechanisms by which MFAP5 binds Notch and instigates downstream signaling are yet unknown. Of note, to-date there have been no accounts of MFAP5 influencing immune processes, and its connection to human disease has not been established. Most importantly, a causal relationship between MFAP5 and the suppression of inflammation has never been previously demonstrated nor hypothesized.

The present disclosure demonstrates a causal role of exogenously delivered MFAP5 in the treatment of immune diseases by an anti-inflammatory mechanism. One specific example is presented in colitis. The present invention therefore provides MFAP5 polypeptides, and various related compositions and methods.

PEA

Proenkephalin A (Penk; a.k.a. PEA) is the precursor of the enkephalin opioid peptides and is proteolytically processed to yield Met-enkephalin, Leu-enkephalin, Met-enkephalin-Arg-Phe, Metenkephalin-Arg-Gly-Leu, enkelytin and PENK-derived peptides (Table 1) (Metz-Boutigue, Kieffer et al. 2003).

TABLE 1
Products of proenkephalin (PENK)

| Name | Amino Acid Sequence | MW (da) |
|---|---|---|
| Met-enkephalin | Tyr-Gly-Gly-Phe-Met (SEQ ID NO. 14) | 576 |
| Leu-enkephalin | Tyr-Gly-Gly-Phe-Leu (SEQ ID NO. 15) | 554 |
| Peptide F (human) | Tyr-Gly-Gly-Phe-Met-Lys-Lys-Met-Asp-Glu-Leu- | 3846 |
| | Tyr-Pro-Met-Glu-Pro-Glu-Glu-Glu-Ala-Asn-Gly-Ser- | |
| | Glu-ne-Leu-Ala-Lys-Arg-Tyr-Gly-Gly-Phe-Met (SEQ | |
| | ID NO. 16) | |
| Peptide E (human and | Tyr-Gly-Gly-Phe-Met-Arg-Arg-Val-Gly-Arg-Pro-Glu- | 3157 |
| bovine) | Trp-Trp-Met-Asp-Tyr-Gln-Lys-Arg-Tyr-Gly-Gly-Phe- | |
| | Leu (SEQ ID NO. 17) | |
| Peptide B (human) | Phe-Ala-Glu-Ala-Leu-Pro-Ser-Asp-Glu-Glu-Gly-Glu- | 3655 |
| | Ser-Tyr-Ser-Lys-Glu-Val-Pro-Glu-Met-Glu-Lys-Arg- | |
| | Tyr-Gly-Gly-Phe-Met-Arg-Phe (SEQ ID NO. 18) | |

Cleavage of PENK by proteases takes place at dibasic, lysine-lysine, amino acid residues. It is expressed in specific sites in the brain and certain cells of the adrenal medulla and the immune system, which is increased at the gene expression level in response to inflammation (Behar, Ovadia et al. 1994). The physiological relevance of the responsiveness of the PENK gene in these sites remains unclear. The concentration of PENK-derived peptides in the unstimulated bovine adrenal medulla exceeds 200 μg per gram of granule protein in secretory vesicles of chromaffin cells (Hook, Noctor et al. 1999). It is worthwhile to note that, except in the setting of meningococcemia, inflammation of the adrenal gland is a medical rarity. Peptides with antimicrobial activity but no known neuropeptide function have been identified in the adrenal medullary chromaffin cell discharge. They include enkelytin and peptide B from PENK (Goumon, Lugardon et al. 1998). The present disclosure demonstrates that a human mesenchymal cell basally secretes PENK and/or PENK-derived peptides, and upregulates this secretion under inflammatory stimuli.

Investigators have proposed that PENK contains neuropeptides, antibacterial peptides, and immune stimulatory peptides (Salzet and Tasiemski 2001). The effects of enkephalins on various activities of immune cells (chemotaxis, cytotoxicity, immunoglobulin synthesis), which bear opioid receptors, have been described. However, there have been no reports of PENK altering cytokine secretion from immune cells when administered directly in a purified form (Sharp, Roy et al. 1998). One study using PENK-deficient mice reported that T cells could produce IFN-γ and TNF-α but not IL-4 or IL-10 (Weir, McNeill et al. 2006). However, the study did not show a causal link between PENK and these cytokines.

The present disclosure demonstrates a causal role of exogenously delivered PENK in the treatment of immune diseases by an anti-inflammatory mechanism. One specific example is presented in colitis. The present invention therefore provides LGALS3BP polypeptides, and various related compositions and methods

HAPLN1

HAPLN1 (a.k.a. Cartilage-linking protein 1, Cartilage-link protein, CRTL1, Hyaluronan and proteoglycan link protein 1, Proteoglycan link protein) is a glycosylated protein associated with the extracellular matrix (ECM). It is one of a family of four HAPLN molecules, all implicated in the structural formation of extracellular matrix (Spicer, Joo et al. 2003). Members of the family of HAPLN proteins are similar in structure, and share anywhere from 45-52% homology. HAPLN1 also shares significant homology with versican (a.k.a. chondroitin sulfate proteoglycan core protein 2 (CSPG2), chondroitin sulfate proteoglycan 2 (CSPG2) and PG-M). HAPLN1 (a.k.a. Hyaluronan and proteoglycan link protein 1; cartilage link protein; versican core protein) is a link protein that aggregates hyaluronan (HA) and chondroitin sulfate proteoglycans (CSPG) in a 1:1:1 ratio. These HA-CSPG aggregates have been reported to bind to CD44, EGF receptors, sulfated glycolipids, tenascins, fibulins, and neural cell adhesion molecule (Neame, Christner et al. 1986; Barta, Deak et al. 1993). The protein is organized with an N-terminal signal sequence followed by an Ig domain (binds to CSPG), and two consecutive proteoglycan tandem repeat regions.

HAPLN1 is reported to be restricted in expression to the small intestine and placenta primarily. HAPLN1 has been shown to be significantly upregulated in the context of certain cancers, and overexpression of the molecule has been linked with tumorigenicity of mesothelioma and invasiveness of breast cancer (Auvinen, Tammi et al. 2000; Ivanova, Goparaju et al. 2009). The present disclosure describes HAPLN1 expression and production in human mesenchymal cells derived from the bone marrow. HAPLN1, in addition to HAPLN2, HAPLN3 and HAPLN4, have been shown to share structural characteristics to the anti-inflammatory molecule TSG-6 (Blundell, Mahoney et al. 2003). However, no reports exist that demonstrate any association between HAPLN1 and immune processes. In particular, no association has been made between HAPLN1 and a reduction in IFN-γ from stimulated leukocytes as is described herein.

In some embodiments, provided polypeptides have immunomodulatory properties. In some embodiments, provided polypeptides are characterized by an ability, when contacted with mammalian leukocytes in culture, to alter production of at least one pro-inflammatory or anti-inflammatory agent by the mammalian leukocytes. In some embodiments, mammalian leukocytes are leukocytes that have been stimulated by a pro-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, mammalian leukocytes are leukocytes that have been stimulated by an anti-inflammatory mediator (for example, but not limited to, steroids, non-steroidal anti-inflammatory drugs, interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor, and/or combinations thereof). In some embodiments, mammalian leukocytes are naïve leukocytes in that that they have not been stimulated by a pro-inflammatory mediator. In some embodiments, mammalian leukocytes are naïve leukocytes in that that they have not been stimulated by an anti-inflammatory mediator. In some such embodiments, production is altered at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is altered at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments, production is increased. In some such embodiments, production is inhibited. In some such embodiments the at least one agent is a pro-inflammatory agent. In some such embodiments, the at least one agent is an anti-inflammatory agent. In some such embodiments, the at least one pro-inflammatory agent is selected from the group consisting of interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18 and interleukin-8, and combinations thereof. In some such embodiments, the at least one anti-inflammatory agent is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof; in some such embodiments, the at least one anti-inflammatory agent is or includes IL-10.

In some embodiments, provided factors are characterized in that, when one or more is/are administered to colitic mice, one or more features of their colitis is/are attenuated.

In some embodiments, provided factors are characterized in that, consistent with studies of MSC activities (see, for example, (Djouad, Plence et al. 2003; Liu, Lu et al. 2004) which report on suppression of MLRs in xenogeneic cultures), they exert their effects across species barriers.

### Antibodies and Receptors

Having identified and provided particular marrow stromal cell factors, the present invention provides antibodies and receptors that bind specifically to such factors (e.g., to include one or more products of GALNT1, LGALS3BP, MFAP5, HAPLN¹, and/or PENK genes). For example, in some embodiments, antibodies and/or receptors bind specifically to provided marrow stromal cell factors such as GALNT1 polypeptides, nucleic acids encoding them (and/or complements of such nucleic acids); LGALS3BP polypeptides, nucleic acids encoding them (and/or complements of such nucleic acids); MFAP5 polypeptides, nucleic acids encoding them (and/or complements of such nucleic acids); PENK polypeptides, nucleic acids encoding them (and/or complements of such nucleic acids); HAPLN1 polypeptides, nucleic acids encoding them (and/or complements of such nucleic acids); and/or combinations thereof. In many embodiments, provided antibodies bind specifically to polypeptides.

The present invention also provides compositions containing such antibodies and/or receptors (individually or together with provided polypeptides), methods of identifying, characterizing, and/or producing such antibodies and/or receptors, and/or methods of using such antibodies and/or receptors (e.g., in research, diagnostic, and/or therapeutic applications).

### Pharmaceutical Compositions

In general, a pharmaceutical composition comprises an active agent (e.g., a provided marrow stromal cell factor and/or antibody and/or receptor thereto) and one or more pharmaceutically acceptable carriers or excipients. Typically, the active agent is present in a therapeutically effective amount.

In some embodiments of the present invention, a pharmaceutical composition comprises a provided marrow stromal cell factor (and/or antibody and/or receptor thereto) in an amount sufficient to alter cytokine production by leukocytes in culture. For example, in some embodiments, a pharmaceutical composition comprises a provided marrow stromal cell factor (and/or antibody and/or receptor) in an amount sufficient to alter production of at least one inflammatory or anti-inflammatory agent by mammalian leukocytes. In some such embodiments, production is altered at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is altered at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments, production is increased. In some such embodiments, production is inhibited. In some such embodiments the at least one cytokine is a pro-inflammatory cytokine. In some such embodiments, the at least one cytokine is an anti-inflammatory agent. In some such embodiments, the at least one pro-inflammatory agent is selected from the group consisting of interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18 and interleukin-8, and combinations thereof. In some such embodiments, the at least one anti-inflammatory agent is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof in some such embodiments, the at least one anti-inflammatory agent is or includes IL-10.

In some embodiments of the present invention, a pharmaceutical composition comprises two or more provided polypeptide products of the genes in Table 2 in combination (and/or antibody and/or receptor thereto) in an amount sufficient to alter cytokine production by leukocytes in culture.

TABLE 2
Representative sequences of GALNT1, LGALS3BP, MFAP5 and PENK polypeptides

| Protein | Exemplary Sequence | GenBank | NCBI RefSeq |
|---|---|---|---|
| GALNT1 | MRKFAYCKVVLATSLIWVLLDMFLLL | AAH47746.1, | NP_065207.2, |
| | YFSECNKCDEKKERGLPAGDVLEPVQ | AAH38440.1 | NP_001153876.1, |
| | KPHEGPGEMGKPVVIPKEDQEKMKEM | | NP_038842.3 |
| | FKINQFNLMASEMIALNRSLPDVRLEG | | |
| | (SEQ ID NO. 19) | | |
| LGALS3BP | MTPPRLFWVWLLVAGTQGVNDGDMR | CAM23109.1, | NP_005558.1, |
| | LADGGATNQGRVEIFYRGQWGTVCDN | AAA36193.1 | NP_035280.1. |
| | LWDLTDASVVCRALGFENATQALGRA | | |
| | AFGQGSGPIMLDEVQCTGTEASLADCK | | |
| | SLGWLKSNCRHERDAGVVCTNETRST | | |
| | HTLDLSRELSEALGQIFDSQRGCDLSIS | | |
| | VNVQGEDALGFCGHTVILTANLEAQA | | |
| | LWKEPGSNVTMSVDAECVPMVRDLLR | | |
| | YFYSRRIDITLSSVKCFHKLASAYGAR | | |
| | QLQGYCASLFAILLPQDPSFQMPLDLY | | |
| | AYAVATGDALLEKLCLQFLAWNFEAL | | |
| | TQAEAWPSVPTDLLQLLLPRSDLAVPS | | |
| | ELALLKAVDTWSWGERASHEEVEGLV | | |
| | EKIRFPMMLPEELFELQFNLSLYWSHE | | |
| | ALFQKKTLQALEFHTVPFQLLARYKGL | | |
| | NLTEDTYKPRIYTSPTWSAFVTDSSWS | | |
| | ARKSQLVYQSRRGPLVKYSSDYFQAPS | | |
| | DYRYYPYQSFQTPQHPSFLFQDKRVS | | |
| | WSLVYLPTIQSCWNYGFSCSSDELPVL | | |
| | GLTKSGGSDRTIAYENKALMLCEGLFV | | |
| | ADVTDFEGWKAATPSALDTNSSKSTSS | | |
| | FPCPAGHFNGFRTVIRPFYLTNSSGVD | | |
| | (SEQ ID NO. 20) | | |
| MFAP5 | MSLLGPKVLLFLAAFIITSDWIPLGVNS | AAH05901.1, | NP_056591.1, |
| | QRGDDVTQATPETFTEDPNLVNDPAT | AAA96752.1, | NP_003471.1 |
| | DETVLAVLADIAPSTDDLASLSEKNTT | AAD53950.1 | |
| | AECWDEKFTCTRLYSVHRPVKQCIHQ | | |
| | LCFTSLRRMYIVNKEICSRLVCKEHEA | | |
| | MKDELCRQMAGLPPRRLRRSNYFRLP | | |
| | PCENVDLQRPNGL (SEQ ID NO. 21) | | |
| PENK | MARFLTLCTWLLLLGPGLLATVRAECS | CAG46627.1, | NP_001129162.1, |
| | QDCATCSYRLVRPADISFLACVMECEG | CAG46607.1, | NP_006202.1, |
| | KLPSLKIWETCKELLQLSRPELPQDGTS | AAI07707.1 | NP_001002927.1 |
| | TLRENSKPEESHLLAKRYGGFMKRYG | | |
| | GFMKKMDELYPMEPEEEANGSEILAK | | |
| | RYGGFMKKDAEEDDSLANSSDLLKEL | | |
| | LETGDNRERSHHQDGSDNEEEVSKRY | | |
| | GGFMRGLKRSPQLEDEAKELQKRYGG | | |
| | FMRRVGRPEWWMDYQKRYGGFLKRF | | |
| | AEALPSDEEGESYSKEVPEMEKRYGGF | | |
| | MRF (SEQ ID NO. 22) | | |
| HAPLN1 | MKSLLLLVLISICWADHLSDNYTLDHD | AAH57808.1 | NP_001875.1 |
| | RAIHIQAENGPHLLVEAEQAKVFSHRG | AAI51456.1 | NP_038528.3 |
| | GNVTLPCKFYRDPTAFGSGIHKIRIKWT | | |
| | KLTSDYLKEVDVFVSMGYHKKTYGG | | |
| | YQGRVFLKGGSDSDASLVITDLTLEDY | | |
| | GRYKCEVIEGLEDDTVVVALDLQGVV | | |
| | FPYFPRLGRYNLNFHEAQQACLDQDA | | |
| | VIASFDQLYDAWRGGLDWCNAGWLS | | |
| | DGSVQYPITKPREPCGGQNTVPGVRNY | | |
| | GFWDKDKSRYDVFCFTSNFNGRFYYLI | | |
| | HPTKLTYDVAVQACLNDGAQIAKVGQ | | |
| | IFAAWKILGYDRCDAGWLADGSVRYP | | |
| | ISRPRRRCSPTEAAVRFVGFPDKKHKL | | |
| | YGVYCFRAYN (SEQ ID NO. 23) | | |

For example, in some embodiments, a pharmaceutical composition comprises a combination of two or more provided polypeptides (and/or antibody and/or receptor) in an amount sufficient to alter production of at least one inflammatory or anti-inflammatory agent by mammalian leukocytes. In some embodiments, mammalian leukocytes are leukocytes that have been stimulated by a pro-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, mammalian leukocytes are leukocytes that have been stimulated by an anti-inflammatory mediator (for example, but not limited to, steroids, non-steroidal anti-inflammatory drugs, interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor, and/or combinations thereof). In some embodiments, mammalian leukocytes are naïve leukocytes in that that they have not been stimulated by a pro-inflammatory mediator. In some embodiments, mammalian leukocytes are naïve leukocytes in that that they have not been stimulated by an anti-inflammatory mediator. In some such embodiments, production is altered at least 10%, at least 20%, at least 30%, at least 40%, at least 50%, at least 100%, at least 150%, at least 200%, at least 250%, at least 300%, at least 350%, at least 400%, at least 450%, at least 500%, or more. In some such embodiments, production is altered at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, or more. In some such embodiments, production is increased. In some such embodiments, production is inhibited. In some such embodiments the at least one agent is a pro-inflammatory agent. In some such embodiments, the at least one agent cytokine is an anti-inflammatory agent. In some such embodiments, the at least one pro-inflammatory agent is selected from the group consisting of interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18 and interleukin-8, and combinations thereof. In some such embodiments, the at least one anti-inflammatory molecule is selected from the group consisting of interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor and combinations thereof; in some such embodiments, the at least one anti-inflammatory molecule is or includes IL-10.

In some embodiments, pharmaceutical compositions comprise a provided factor and/or a combination of two or more (and/or antibody and/or receptor thereto) in an amount such that, when the pharmaceutical compositions are administered to colitic mice, one or more features of their colitis is/are attenuated.

In some embodiments, pharmaceutical compositions comprise a provided factor and/or a combination of two or more (and/or receptor thereto) at concentrations comparable to those at which such factors (and/or receptors) are naturally found in human serum.

In some embodiments, pharmaceutical compositions comprise a provided factor and/or a combination of two or more (and/or receptor thereto) in an amount within two orders of magnitude of 10 μg/mL.

Pharmaceutical compositions in accordance with the present invention may be formulated for any appropriate route of administration. For example, compositions may be formulated for intravenous, parenteral, subcutaneous, intramuscular, intracranial, intraorbital, ophthalmic, intraventricular, intracapsular, intraspinal, intracistemal, intraperitoneal, intranasal, or aerosol administration. In some embodiments, pharmaceutical compositions are formulated for oral delivery. In some embodiments, pharmaceutical compositions are formulated for parenteral delivery. For treatment of rheumatoid arthritis, in particular, intra-articular administration is specifically contemplated along with other appropriate routes.

Pharmaceutical compositions may be in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc.

Methods and agents well known in the art for making formulations are described, for example, in “Remington's Pharmaceutical Sciences,” Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes.

If desired, slow release or extended release delivery systems may be utilized. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

As described herein, pharmaceutical compositions are formulated to contain a dose of active agent appropriate to the effect to be achieved and are typically administered in unit dosage form. An effective quantity of the purified molecules or molecular mixtures is employed to treat the diseases or conditions described herein. The exact dosage of a molecule or molecular mixture may be dependent, for example, upon the age and weight of the recipient, the route of administration, and the severity and nature of the disease or condition to be treated. In general, the dosage selected should be sufficient to prevent, ameliorate, or treat the disease or condition, or one or more symptoms thereof, without producing significant toxic or undesirable side effects.

### Uses

Marrow stromal cell factors (e.g., polypeptides as described herein), antibodies and receptors thereto, and compositions as described herein have a variety of uses, many of which will be readily apparent to those of ordinary skill in the art reading the present disclosure. Such uses include various research-related uses, diagnostic uses, and/or therapeutic uses.

To give but a few examples, now that particular marrow stromal cell factors (e.g., polypeptides as described herein) polypeptides have been identified in accordance with the present invention, those of ordinary skill in the art will appreciate that they can be used as reagents against which the performance or identity of other factors is compared.

Alternatively or additionally, antibodies and receptors to such factors can be used to characterize the factors, including for example measuring levels (e.g., levels of one or more of provided factors in different patients, organs, tissues, and/or cell types, etc.), affinities (e.g., binding affinities, etc.). In some embodiments, characterization assays are performed in vivo. In some embodiments, characterization assays are performed in vitro. In some embodiments, characterization assays are performed using cells that have been pre-stimulated with at least one pro-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, characterization assays are performed using cells that have not been pre-stimulated with at least one pro-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, characterization assays are performed using cells that have been pre-stimulated with at least one anti-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, characterization assays are performed using cells that have not been pre-stimulated with at least one anti-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof).

Still further, the present invention provides systems for detecting levels of provided factors, for example to assess whether a sample containing such factors has therapeutic potential. In some embodiments, a sample to be assessed comprises one or more marrow stromal cells. In some embodiments, a sample to be assessed is or comprises a pharmaceutical formulation. For example, in some embodiments, the present invention provides systems of evaluating and/or confirming quality of a proposed therapeutic sample by detecting levels of one or more provided factors in the sample and determining, for example based on the detected level, that the sample is or is not likely to have therapeutic potential because it does or does not contain a requisite level of marrow stromal cell factor as described herein. In some embodiments, detection assays are performed using cells that have been pre-stimulated with at least one pro-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, detection assays are performed using cells that have not been pre-stimulated with at least one pro-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, detection assays are performed using cells that have been pre-stimulated with at least one anti-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, detection assays are performed using cells that have not been pre-stimulated with at least one anti-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof).

Therapeutic uses of provided marrow stromal cell factors (and/or antibodies and/or receptors thereto) and/or compositions comprising them (and/or antibodies and/or receptors to them) include, for example, uses in medicine. For example, provided compositions may be administered to a subject suffering from or susceptible to one or more diseases, disorders, or conditions associated with inflammation. In some such embodiments, the subject is suffering from or susceptible to one or more diseases, disorders or conditions presented in Table 3:

Table 3
List of Inflammatory Diseases

Multiple sclerosis, type 1 diabetes, rheumatoid arthritis, uveitis, autoimmune thyroid disease,
scleroderma, autoimmune lymphoproliferative disease (ALPS), demyelinating disease,
autoimmune encephalomyelitis, autoimmune gastritis (AIG), autoimmune glomerular disease,
inflammatory bowel disease including Crohn's Disease and ulcerative colitis, psoriasis, uveitis,
Celiac disease, pernicious anemia, Srojen's syndrome, Hashimoto's thyroiditis, Graves' disease,
systemic lupus erythamatosis, acute disseminated encephalomyelitis, Addison's disease,
Ankylosing spondylitis, Antiphospholipid antibody syndrome, Guillain-Barre syndrome,
idiopathic thrombocytopenic purpura, Goodpasture's syndrome, Myasthenia gravis, Pemphigus,
giant cell arteritis, aplastic anemia, autoimmune hepatitis, Kawaski's disease, mixed connective
tissue disease, Ord' throiditis, polyarthritis, primary biliary sclerosis, Reiter's syndrome,
Takaysu's arteritis, vitiligo, warm autoimmune hemolytic anemia, Wegener's granulomatosis,
Chagas' disease, chronic obstructive pulmonary disease, sarcoidosis, acute respiratory distress
syndrome, systemic inflammatory response syndrome, multiple organ dysfunction syndrome,
sepsis, acute pancreatitis, acute liver failure, acute-on-chronic liver failure, chronic liver failure,
acute renal failure, end stage renal disease, chronic renal failure, nephrotic syndrome, nephritic
syndrome, focal segmental glomerulosclerosis, glomerulonephritis, acute tubular necrosis, lupus
nephritis, diabetic nephritis, interstitial nephritis, acute-on-chronic renal failure, aplastic anemia,
fanconi anemia, hemolytic anemia, iron-deficient anemia, pernicious anemia, sickle cell anemia,
hemochromatosis, hemophilia, idiopathic thrombocytopenic purpura, polycythemia vera, rh
incompatibility, thalassemias, thrombocytopenia, thrombocythemia, thrombocytosis,
thrombophlebitis, thrombotic thrombocytopenic purpura, Von Willebrand disease, leukemia,
lymphatic filariasis, Anemia From Excessive Bleeding, Anemia of Chronic Disease,
Autoimmune Hemolytic Anemia, Hemoglobin C, S-C, and E Diseases, Vitamin Deficiency
Anemia, Disseminated Intravascular Coagulation (DIC), Henoch-Schönlein Purpura, Hereditary
Hemorrhagic Telangiectasia, Thrombophilia, Acute Lymphocytic Leukemia (ALL), Acute
Myelocytic Leukemia (AML), Chronic Lymphocytic Leukemia (CLL), Chronic Myelocytic
Leukemia (CML), Hodgkin's Disease, Non-Hodgkin's Lymphomas, Myelofibrosis,
Macroglobulinemia, Monoclonal Gammopathies of Undetermined Significance (MGUS),
Multiple Myeloma, Lymphocytopenia, Neutropenia, Neutrophilic Leukocytosis, Chronic Pain,
Migraine, Multiple Abortions, Asthma, Myocardial Infarction, Atherosclerosis, Cancer, Type 2
Diabetes, Obesity, Psoriatic Arthritis, Polyarticular Juvenile Idiopathic Arthritis, Acute
inflammatory demyelinating polyradiculopathy (Guillain Barre Syndrome), Chronic
inflammatory demyelinating polyradiculopathy, Nerve injury, Graft vs. host disease, Behcet's
disease, Rheumatoid vasculitis, Churg Strauss Syndrome, Takayasu's arteritis, Giant cell
arteritis, Polyarteritis nodosa, Cryoglobulinemic vasculitis, Congestive heart failure, Acute Lung
Injury, and/or combinations thereof.

In some such embodiments, the subject is suffering from or susceptible to one or more diseases, disorders, or conditions selected from the group consisting of rheumatoid arthritis, type I and type II diabetes, ulcerative colitis, Crohn's disease, celiac disease, multiple sclerosis, myocardial infarction, neoplasm, chronic infectious disease, systemic lupus erythematosus, acute kidney injury, sepsis, multiple organ dysfunction syndrome, acute liver failure, chronic liver failure, chronic kidney failure, pancreatitis, Grave's disease, and combinations thereof.

In some embodiments, provided marrow stromal cell factors (and/or antibodies and/or receptors thereto) and/or compositions comprising them (and/or antibodies and/or receptors to them) are used to increase production of one or more anti-inflammatory agents in a mammal. In some embodiments, provided factors (and/or antibodies and/or receptors thereto) and/or compositions comprising them (and/or antibodies and/or receptors to them) are used to decrease production of one or more inflammatory agents in a mammal.

In some embodiments, provided marrow stromal cell factors (and/or antibodies and/or receptors thereto) and/or compositions comprising them (and/or antibodies and/or receptors to them) are administered to a patient who has previously been and/or is currently being treated with at least one pro-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, such a patient's leukocytes have been pre-stimulated with the at least one pro-inflammatory mediator prior to treatment with one or more provided marrow stromal cell factors (and/or antibodies and/or receptors thereto) and/or compositions comprising them (and/or antibodies and/or receptors to them). In some embodiments, such a patient's leukocytes have been not pre-stimulated with the at least one pro-inflammatory mediator prior to treatment with one or more provided marrow stromal cell factors (and/or antibodies and/or receptors thereto) and/or compositions comprising them (and/or antibodies and/or receptors to them). In some embodiments, provided marrow stromal cell factors (and/or antibodies and/or receptors thereto) and/or compositions comprising them (and/or antibodies and/or receptors to them) are administered to a patient who has not previously been and/or is not currently being treated with at least one pro-inflammatory mediator (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, such a patient's leukocytes have been not pre-stimulated with the at least one pro-inflammatory mediator prior to treatment with one or more provided marrow stromal cell factors (and/or antibodies and/or receptors thereto) and/or compositions comprising them (and/or antibodies and/or receptors to them).

In some embodiments, provided marrow stromal cell factors (and/or antibodies and/or receptors thereto) and/or compositions comprising them (and/or antibodies and/or receptors to them) are administered to a patient who has previously been and/or is currently being treated with at least one anti-inflammatory mediator (for example, but not limited to, steroids, non-steroidal anti-inflammatory drugs, interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor, and/or combinations thereof). In some embodiments, such a patient's leukocytes have been pre-stimulated with the at least one anti-inflammatory mediator prior to treatment with one or more provided marrow stromal cell factors (and/or antibodies and/or receptors thereto) and/or compositions comprising them (and/or antibodies and/or receptors to them). In some embodiments, such a patient's leukocytes have been not pre-stimulated with the at least one anti-inflammatory mediator prior to treatment with one or more provided marrow stromal cell factors (and/or antibodies and/or receptors thereto) and/or compositions comprising them (and/or antibodies and/or receptors to them). In some embodiments, provided marrow stromal cell factors (and/or antibodies and/or receptors thereto) and/or compositions comprising them (and/or antibodies and/or receptors to them) are administered to a patient who has previously been and/or is currently being treated with at least one anti-inflammatory mediator (for example, but not limited to, steroids, non-steroidal anti-inflammatory drugs, interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor, and/or combinations thereof). In some embodiments, such a patient's leukocytes have not been pre-stimulated with the at least one anti-inflammatory mediator prior to treatment with one or more provided marrow stromal cell factors (and/or antibodies and/or receptors thereto) and/or compositions comprising them (and/or antibodies and/or receptors to them). In some embodiments, such a patient's leukocytes have been not pre-stimulated with the at least one anti-inflammatory mediator prior to treatment with one or more provided marrow stromal cell factors (and/or antibodies and/or receptors thereto) and/or compositions comprising them (and/or antibodies and/or receptors to them).

### Combination Therapy

As will be clear to those of ordinary skill in the art, pharmaceutical compositions as described herein may be employed in combination therapy. In some embodiments, two or more agents utilized in combination are administered in a single composition; in some embodiments, two or more agents utilized in combination are administered in separate compositions.

In some embodiments, as described herein, individual marrow stromal cell factors (e.g., polypeptides as described herein) polypeptides (and/or other provided agents or compositions) are administered in combination with one another (e.g., any combination of one or more of GALNT1 polypeptides, LGALS3BP polypeptides, MFAP5 polypeptides, HAPLN1 polypeptides, PENK polypeptides, nucleic acids encoding any of the foregoing, and/or antibodies against any of the foregoing). In some embodiments, provided polypeptides encoded by genes in Table 2 (and/or other provided agents or compositions) are administered in combination with one another and/or other factors to be used in the treatment of one or more diseases, disorders or conditions. In some embodiments, the one or more diseases, disorders or conditions are characterized by inflammation.

In some embodiments, individual marrow stromal cell factors (e.g., polypeptides as described herein) polypeptides (and/or other provided agents or compositions) are administered in combination with one or more pro-inflammatory mediators (for example, but not limited to, lipopolysaccharide (LPS), DNA, RNA, bacterial products, viral products, non-human products, human products, toxins, chemicals, interleukin-1-α, interleukin-1-β, interleukin-6, tumor necrosis factor-α, leukemia inhibitory factor, interferon-γ, other interferons, oncostatin M, ciliary neurotrophic factor, granulocyte-macrophage colony-stimulating factor, interleukin-11, interleukin-12, interleukin-17, interleukin-18, interleukin-8, and/or combinations thereof). In some embodiments, individual marrow stromal cell factors (e.g., polypeptides as described herein) polypeptides (and/or other provided agents or compositions) are administered in combination with one or more anti-inflammatory mediators (for example, but not limited to, steroids, non-steroidal anti-inflammatory drugs, interleukin-10, TGF-β, interleukin-1 receptor antagonist, interleukin-1 soluble receptor, tumor necrosis factor-α soluble receptors I and II, interleukin-4, interleukin-6, interleukin-11, interleukin-13, interleukin-16, interleukin-18 soluble receptor, atrial natriuretic peptide, interleukin-6 soluble receptor, and/or combinations thereof).

### Exemplification

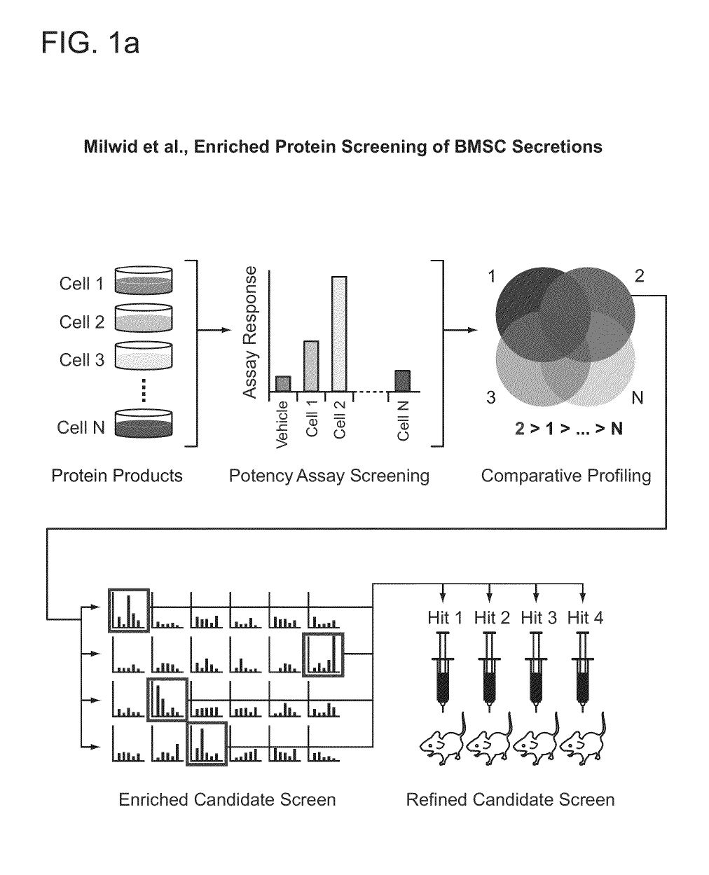

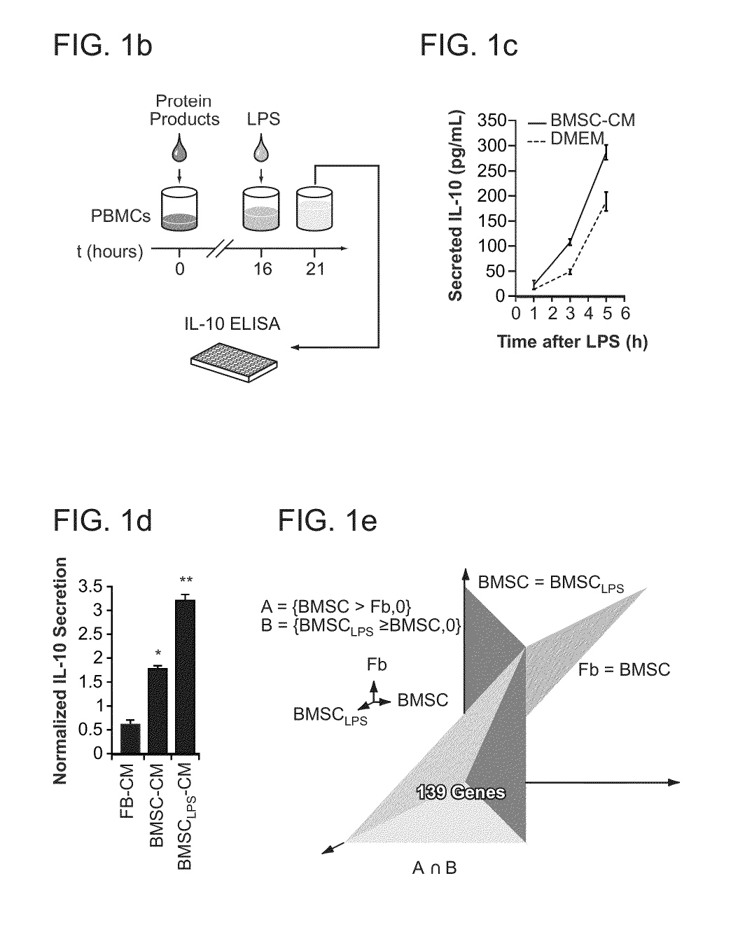

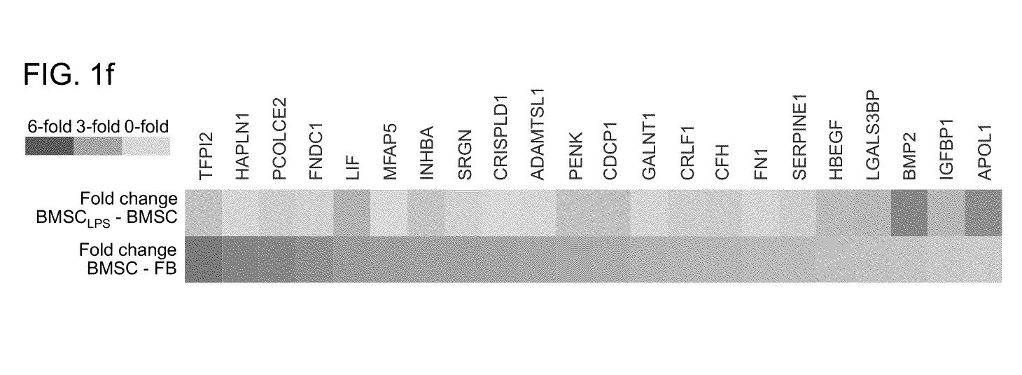

The present inventors have developed new methods for screening proteins expressed by BMSCs. The design of this approach (which can be referred to as “enriched protein screening,” or “EPS”) is shown and described in FIG. 1a.

The following examples describe use of these methods to identify certain factors produced by MSCs and/or demonstrate that certain factors produced by MSCs modulate inflammatory cytokine production from leukocytes in culture, for example at concentrations within two orders of magnitude of 10 μg/mL and/or within an order of magnitude (or otherwise comparable to) those concentrations at which relevant compounds are naturally found in human serum. These examples also demonstrate that these factors can be exogenously delivered in a purified form to protect mice from inflammatory diseases, disorders, or conditions (e.g., TNBS colitis). In one particular example, isolated and purified molecular products of the genes LGALS3BP, MFAP5, GALNT1, CFH, TFPI2, PENK, HAPLN1 and CRLF1 were administered at concentrations within five orders of magnitude, either higher or lower, of 10 μg/mL, and shown to either promote IL-10 production or suppress IFN-γ production in human leukocytes in culture. Four of these purified molecular products, namely from genes LGALS3BP, MFAP5, PENK, and GALNT1, were administered to colitic mice and three of the four, LGALS3BP, PENK, and MFAP5, were shown to protect the mice from developing acute colitis.

### Materials and Methods

### Statistics

Unless otherwise noted, all experiments were repeated in quadruplicate, and all data were assessed for significance using a paired, two-tail Student's T test.

### Cell Culture and Conditioned Medium

BMSCs were isolated, purified, grown and characterized, and fibroblasts grown as described previously (Jiao, Y., Milwid, J. M., Yarmush, M. L. & Parekkadan, B. in Suppression and Regulation of Immune Responses, Vol. 677. (eds. M. Cuturi & I. Anegon) (Humana Press and Springer, Totowa, N.J., USA; 2010); and Parekkadan, B. et al. Mesenchymal stem cell-derived molecules reverse fulminant hepatic failure. PLoS ONE 2 (2007); the contents of both of which are incorporated herein by reference). All BMSCs were used at passage 2-5. Conditioned medium was also collected and concentrated as described previously (van Poll, D. et al. Mesenchymal stem cell-derived molecules directly modulate hepatocellular death and regeneration in vitro and in vivo. Hepatology 47, 1634-1643 (2008); incorporated herein by reference). For BMSC_{LPS} conditioned medium and cells used for gene expression analysis, BMSCs were grown to >80% confluence and rinsed twice with PBS. BMSC or fibroblast expansion medium supplemented with 1 μg/mL LPS (E. coli 0111:B4; Sigma, St. Louis, Mo.) was then added to cells for 24 hours, at which time cells were rinsed with PBS twice again and incubated for an additional 24 hours with serum-free DMEM to produce conditioned medium. By convention, 1× refers to the concentration of conditioned medium achieved when 15 mL of conditioning medium was incubated in the presence of 2×10⁶ cells for 24 hours, collected, and concentrated to a final volume of 1 mL.

### Peripheral Blood Mononuclear Cell Potency Assay

The assay was performed as before (Jiao, Y., Milwid, J. M., Yarmush, M. L. & Parekkadan, B. in Suppression and Regulation of Immune Responses, Vol. 677. (eds. M. Cuturi & I. Anegon) (Humana Press and Springer, Totowa, N.J., USA; 2010); incorporated herein by reference). Approval for the collection of blood from healthy volunteers was obtained from the Institutional Review Board of Massachusetts General Hospital. For the majority of the experiments, the potency assay was terminated at 5 hours for IL-10 analysis.

### Gene Expression Analysis

Gene expression was evaluated using Affymetrix GeneChip® Human Genome U133 Plus 2.0 Arrays (Affymetrix, Santa Clara, Calif.). Array quality was assessed using the R/Bioconductor package (Gentleman, R. et al. Bioconductor: open software development for computational biology and bioinformatics. Genome Biology 5, R80 (2004); incorporated herein by reference). All arrays passed visual inspection and no technical outliers were identified (n=3 arrays per cell type). Raw CEL files were processed using the robust multiarray average (RMA) algorithm (Irizarry, R. et al. Summaries of Affymetrix GeneChip probe level data. Nucleic Acids Research 31, e15 (2003); incorporated herein by reference). To identify genes correlating with the observed phenotypic groups, we used limma (Smyth, G. Linear models and empirical Bayes methods for assessing differential expression in microarray experiments. Statistical Applications in Genetics and Molecular Biology 3, 1027 (2004); incorporated herein by reference) to fit a statistical linear model to the data and then tested for differential gene expression in the contrasts of interest: FB vs. BMSC; BMSC vs. LPS. Results were adjusted for multiple testing using the Benjamini and Hochberg (BH) method (Benjamini, Y. & Hochberg, Y. Controlling the false discovery rate: a practical and powerful approach to multiple testing. Journal of the Royal Statistical Society. Series B (Methodological) 57, 289-300 (1995); incorporated herein by reference), and significance was determined using a False-Discovery-Rate cutoff of less than 1%. All genes identified to be upregulated in BMSCs compared to FBs and either expressed equally or upregulated in BMSC_{LPS} compared to BMSCs were then analyzed by literature review for definitive evidence of production of a secreted protein by human cells to generate the list of genes reported here. Independent analysis conducted in collaboration with a second computational biology facility confirmed these same results with >95% overlap of secreted proteins identified via literature review.

### Recombinant Protein Screen

Recombinant proteins were acquired from a commercial vendor (Abnova, Taipei, Taiwan). The proteins were diluted in PBS and added to the PBMC potency assay to achieve a range of final concentrations spanning ˜1 μg/mL to ˜0.01 ng/mL.

### ELISAs and Western Blots

The ELISA kits used were provided by commercial vendors and were used according to the manufacturers' instructions (IL-10 in cell supernatants: BD, Franklin Lakes, N.J.; LGALS3BP: Abnova, Taipei, Taiwan; IL-10 and TNF-α from animal serum: R&D Systems, Minneapolis, Minn.). To blot for GALNT1, MFAP5 and PENK, FB-CM, BMSC-CM and BMSC_{LPS}-CM were run out using protein gel electrophoresis (Pierce, Rockford, Ill.) followed by blotting using detection antibodies (Sigma, St. Louis, Mo.) applied at a dilution of 1:500 (GALNT1 and MFAP5) or 1:100 (PENK), and corresponding secondary antibodies (anti-rabbit for GALNT1 and MFAP5 and anti-goat for PENK) conjugated with HRP (Sigma, St. Louis, Mo.). For the LGALS3BP ELISA and the MFAP5 blot, 1× conditioned media were used, and for the GALNT1 and PENK blots, 20× conditioned media were used.

### Mass Spectrometry

Mass spectrometry was performed at the Mass Spectrometry Core facility of the Beth Israel Deaconess Medical Center at Harvard Medical School as previously described (Jiang, X., Chen, S., Asara, J. & Balk, S. Phosphoinositide 3-kinase pathway activation in phosphate and tensin homolog (PTEN)-deficient prostate cancer cells is independent of receptor tyrosine kinases and mediated by the p110 and p110 catalytic subunits. Journal of Biological Chemistry 285, 14980 (2010); incorporated herein by reference). 10× samples of BMSC-CM, FB-CM and BMSC_{LPS}-CM were initially separated by SDS-PAGE and bands were excised and trypsin digested for analysis via tandem LC/MS/MS. The false discovery rate (FDR) for peptide identifications was ˜1.5% and less than 0.5% for protein identifications.

### In Vivo Mouse Assay

All procedures were performed in accordance with the animal rights policies of the Massachusetts General Hospital Subcommittee on Research Animal Care. For the sub-lethal LPS model, eight week old female BALB/cJ mice (n≧3) (Jackson Laboratories, Bar Harbor, Me.) were administered an initial dose of either vehicle or one of the experimental therapies IP: 200 μL of saline (vehicle), or 3 μg of protein (e.g., GALNT1, LGALS3BP, MFAP5 or PENK) diluted in 200 μL of saline, or 1 mL of 1×BMSC-CM. 16 hours later, mice received a second dose of either vehicle or therapy in conjunction with a dose of 100 μg of LPS (E. coli 0111:B4; Sigma, St. Louis, Mo.) diluted in physiological saline. 48 hours later, mice were sacrificed and tissue and blood were collected for analysis. Serum was tested for the presence of IL-10 and TNF-α via ELISA and lungs, livers and kidneys of the animals were preserved for hematoxylin and eosin staining. For the lethal LPS model, eight week old female BALB/cJ mice (n≧5) were co-administered a lethal dose of LPS (350 μg LPS in 100 μL physiological saline) and either vehicle (negative control), 5 μg anti-TNF-α (positive control; R&D Systems, Minneapolis, Minn.), 4 μg MFAP5 diluted in 100 μL of physiological saline, or 4 μg of PENK diluted in 100 μL of physiological saline. Mice were monitored for survival for seven days (168 hours).

### Chromatography of BMSC-CM

BMSC-CM was injected into the flow circuit of an AKTA purifier FPLC (GE Healthcare, Buckinghamshire, UK) and set to run at 0.5 mL/min. The injected BMSC-CM was run over either a Superdex 200 size exclusion column (GE Healthcare, Buckinghamshire, UK) or a Mono Q 10/100 GL ion exchange column and fixed-volume fractionation was performed using an AKTA Frac-950 (0.5 mL; GE Healthcare, Buckinghamshire, UK).

### Preconditioning of BMSCs

BMSCs were cultured and expanded until >80% confluent. Culture medium was aspirated and the cells rinsed twice with PBS. Culture medium was added to the cells supplemented with varying concentrations of the following: IFN-γ, TNF-α, IL-6 and IL-1β (R&D Systems, Minneapolis, Minn.); Poly I:C DNA (Invivogen, San Diego, Calif.); and LPS (E. coli 0111:B4; Sigma, St. Louis, Mo.). Cells were incubated in the presence of these additives for 24 hours, followed by aspiration of supernatant, two washes with PBS, and addition of DMEM conditioning medium. DMEM conditioning medium was incubated in the presence of cells for 24 hours when it was collected and concentrated as for BMSC-CM.

### Example 1

### Stimulation of Anti-inflammatory Cytokine Production by MSC Secreted Factors

Preparations were made of marrow stromal cell secreted factors by culturing MSCs in the presence of serum-free DMEM for 24 hours, followed by collection and concentration of the supernatant using a 3-kDa cutoff ultrafiltration membrane to yield what is referred to herein as MSC conditioned medium (MSC-CM). In certain experiments, conditioned medium was prepared from skin fibroblasts or from MSCs that have been stimulated with 1 μg/mL of lipopolysaccharide (LPS) supplemented into MSC growth medium for 24 hours prior to conditioning in serum-free DMEM. The concentration of the conditioned medium was defined by the following nomenclature: 1×MSC-CM corresponds herein to the equivalent of 2×10⁶ MSCs cultured in the presence of serum-free DMEM that was concentrated down to a volume of 1 mL after 24 hours of conditioning. Therefore, 10×MSC-CM herein corresponds to the equivalent of 20×10⁶ cells conditioned and concentrated into a final volume of 1 mL.

Leukocytes were prepared from fresh whole human blood, collected and spun in the presence of Ficoll for 30 minutes at 1500×g. The mononuclear cell layer was transferred to a new tube where it was washed once with RPMI 1640. The mononuclear cells were plated at a density of 1×10⁵ per well of a 96-well plate in 50 μL of RPMI 1640 per well. MSC-CM was then immediately added to each well and allowed to incubate in the presence of the mononuclear cells for 16 hours. After incubation, 50 μL of RPMI 1640 containing 10 μg/mL of LPS was added to each well and allowed to incubate for an additional 5 hours. The resultant supernatants were collected and analyzed via ELISA for human interleukin-10 (IL-10). This procedure of incubating leukocytes with MSC-CM to determine IL-10 stimulating activity will be henceforth referred to as the “IL-10 assay”.

MSC-CM was found to cause a significant increase in the IL-10 production of the cultured leukocytes once stimulated with LPS, as seen in FIG. 1b-1c. As also seen in this Figure, the IL-10 production of the leukocytes incubated with MSC-CM responded in a dose-dependent manner, indicating a positive effect of concentrating the conditioned medium on the potency of the IL-10 response.

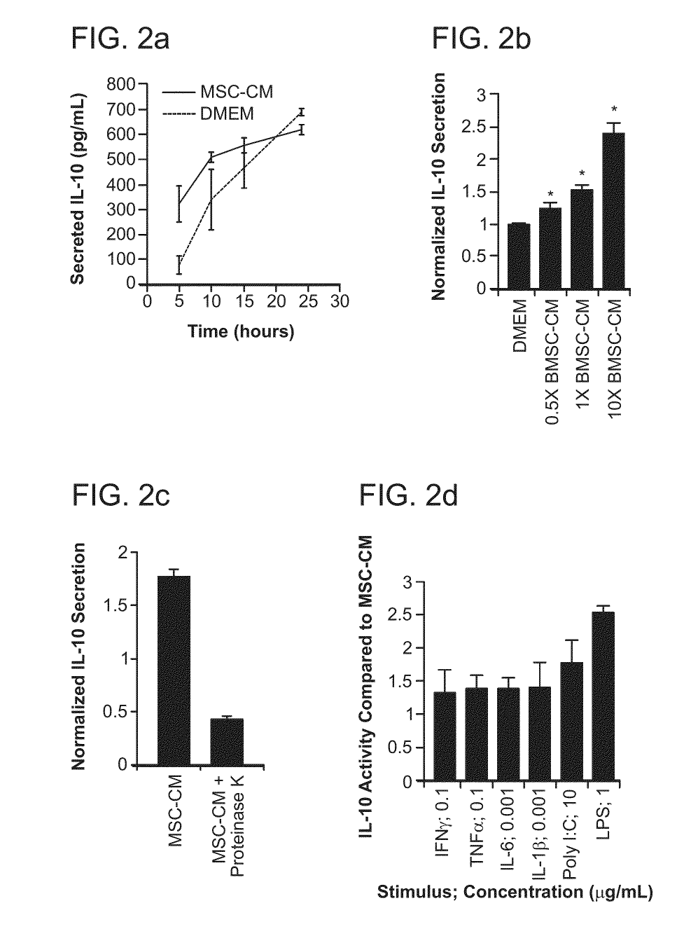

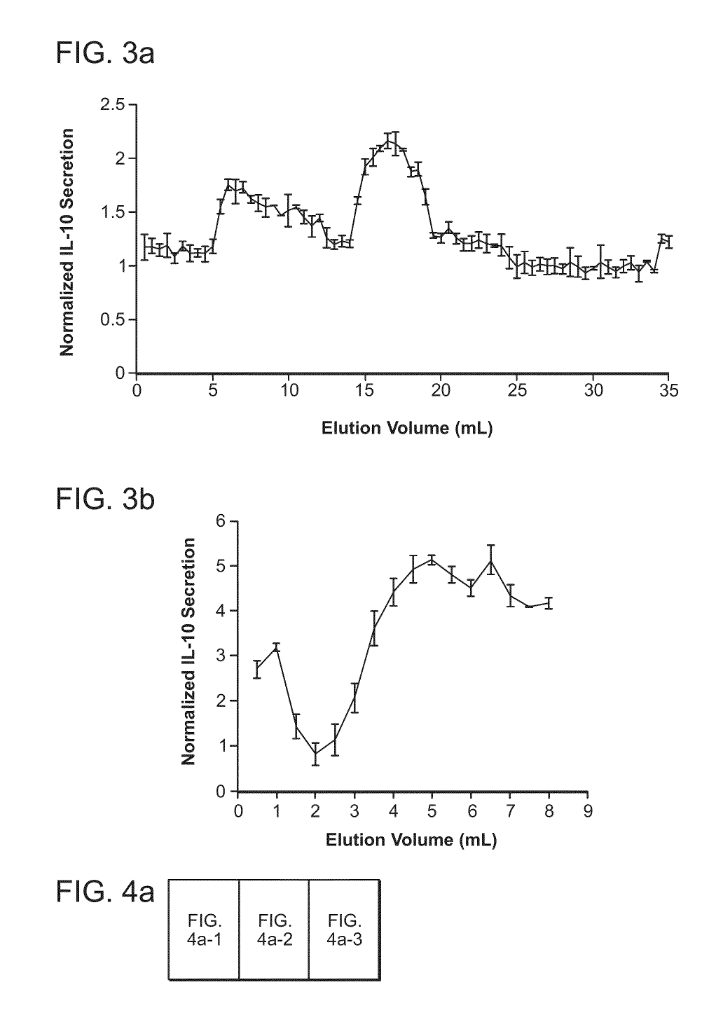

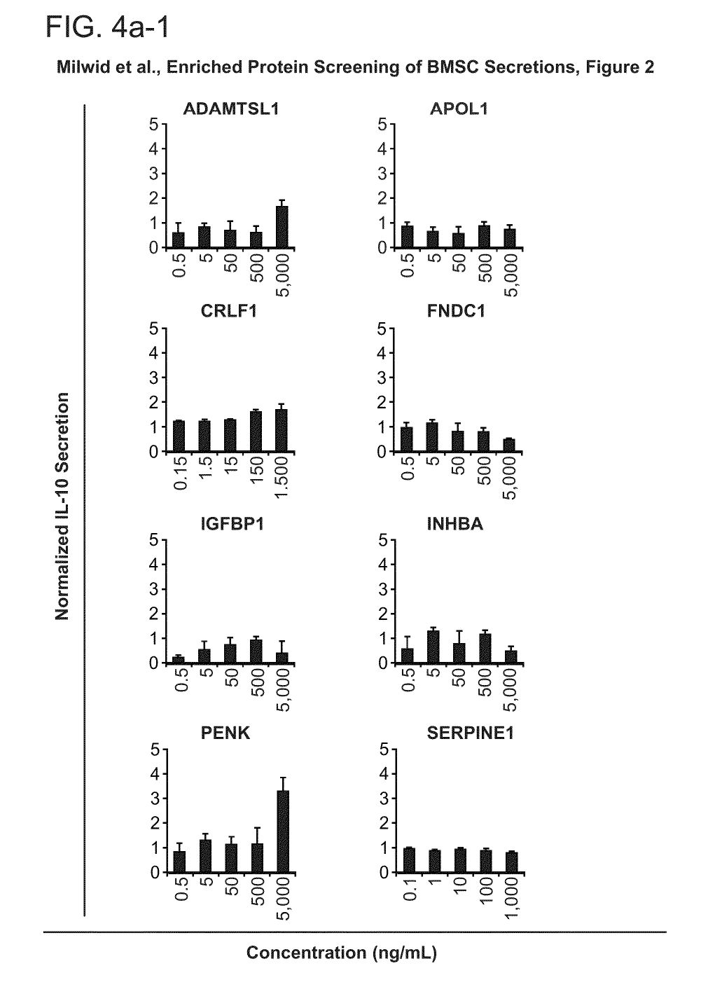

A reproducible in vitro potency assay was used to evaluate bulk anti-inflammatory activity of BMSC-CM (FIG. 1b). These assays demonstrated that LPS-stimulated production of IL-10 by human peripheral blood mononuclear cells (PBMCs) increased significantly when the PBMCs were incubated with BMSC-CM compared to vehicle control (FIG. 1c). Upregulation of IL-10 in PBMCs was time- and dose-dependent (FIG. 2a-b) and was induced by protease-sensitive constituents of BMSC-CM (FIG. 2c).

### Example 2

### Differential Gene Expression Analysis to Determine Active Factors from MSCs

To identify and purify individual factors responsible for this IL-10 response, the inventors performed LC/MS analysis. The inventors successfully identified regions of activity with LC separation based on size and charge (FIG. 3a-b).

To circumvent individually screening hundreds of proteins secreted by BMSCs (van Poll, D. et al. Mesenchymal stem cell-derived molecules directly modulate hepatocellular death and regeneration in vitro and in vivo. Hepatology 47, 1634-1643 (2008); and Chen, L., Tredget, E., Wu, P. & Wu, Y. Paracrine factors of mesenchymal stem cells recruit macrophages and endothelial lineage cells and enhance wound healing. Plos One 3 (2008); both of which are incorporated herein by reference), the inventors developed a comparative gene expression scheme that enabled rational enrichment for candidate proteins that contribute to the anti-inflammatory activity of BMSC-CM. To this end, the inventors first sought to identify a “BMSC signature” in terms of BMSC genes that were associated with IL-10 upregulation in our potency assay. This led to identification of one or more comparative BMSC analogs that were likely to express many similar genes as BMSCs, but lacked activity in the potency assay. CMs from a similar stromal cell (normal human skin fibroblasts (FB)) were tested, and it was determined that FB-CM did not cause a significant increase in IL-10 expression in the potency assay (FIG. 1d). Comparison of gene expression of BMSCs and FBs, yielded a list of ˜500 genes uniquely upregulated in BMSCs. One additional, but essential comparative group, was then included to refine the “BMSC signature”.

The expression signature of BMSCs was then perturbed by establishing conditions under which the activity of the BMSC-CM was enhanced. BMSCs display a number of surface cytokine and toll-like receptors that have been implicated in their immunomodulatory phenotype (Tomchuck, S. L. et al. Toll-like receptors on human mesenchymal stem cells drive their migration and immunomodulating responses. Stem Cells 26, 99-107 (2008); incorporated herein by reference). BMSCs were pre-stimulated with a selection of the cognate ligands for these receptors for 24 hours prior to conditioning. These experiments demonstrated that BMSC-CM from cells that were pre-stimulated with LPS exhibited significantly higher activity in the potency assay (FIG. 1d and FIG. 2d). This information was used to further refine the inventors' analysis by cross-referencing the list of BMSC-specific genes with genes that were maintained at the same level or significantly upregulated by BMSCs stimulated with LPS (BMSC_{LPS}) compared to BMSCs. This comparison revealed 139 genes (FIG. 1e). A literature search was then conducted to determine which of these genes correspond to a secreted protein, yielding a highly enriched set of 22 genes (FIG. 1f).

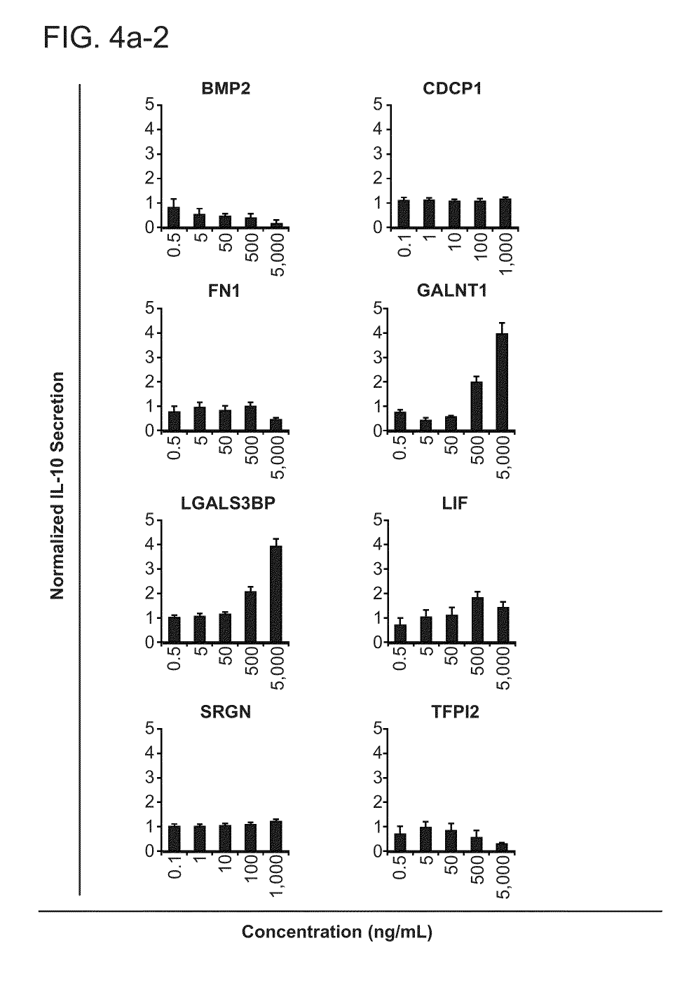

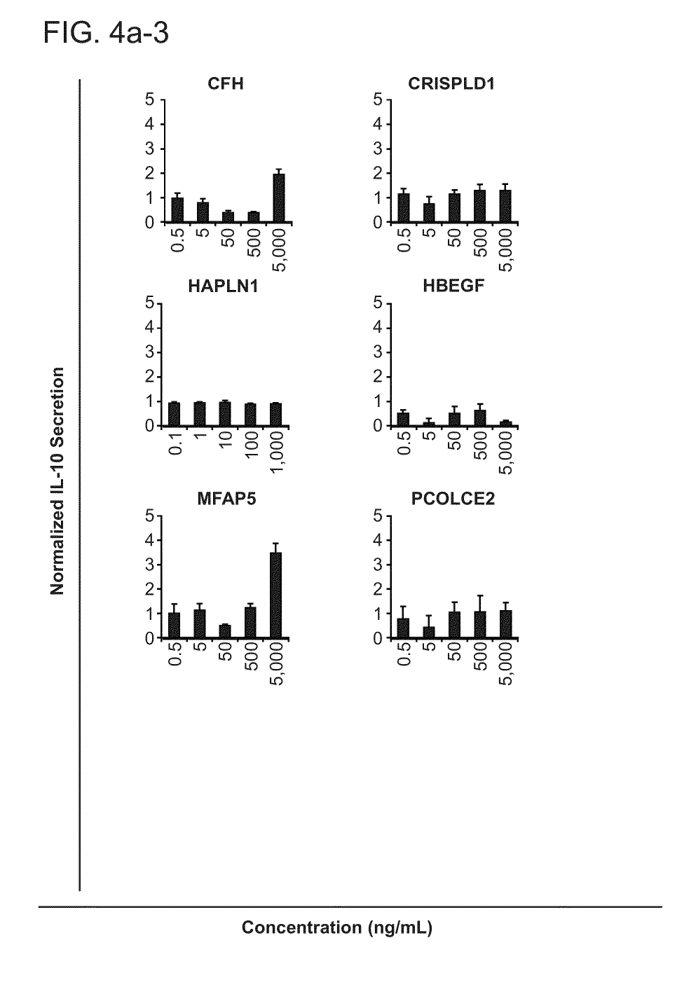

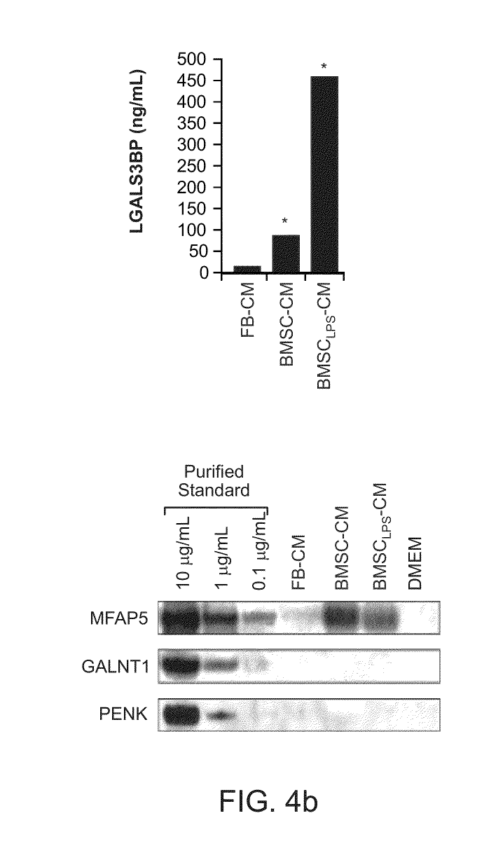

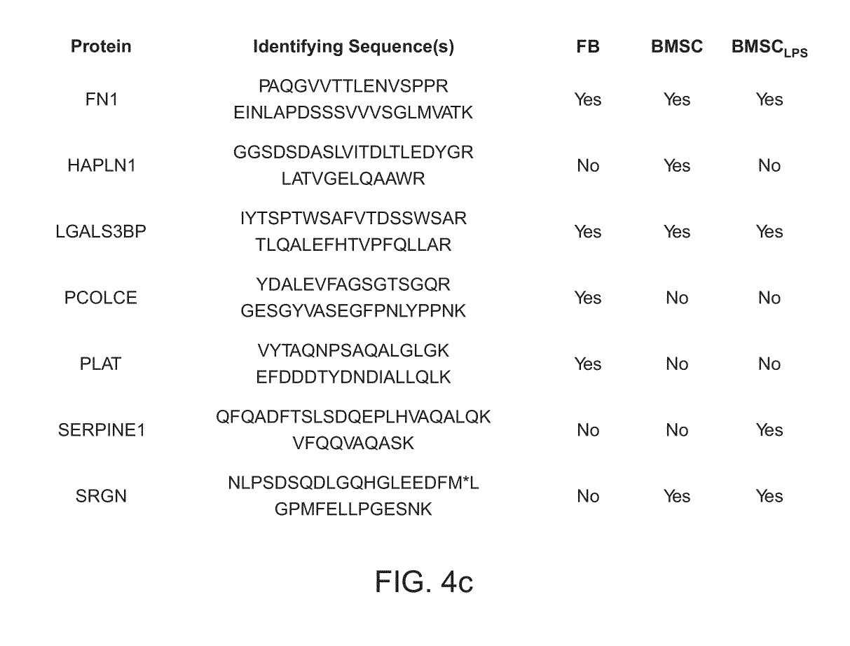

Recombinant molecules were obtained as products of the genes listed in Table 2, which represent the secreted molecule fraction of genes upregulated in LPS-pre-stimulated MSCs compared to MSCs and fibroblasts. The inventors then assembled a purified recombinant protein library corresponding to the 22 candidate genes identified by the enrichment technique. The proteins were individually screened in the in vitro potency assay using a range of physiologically-relevant concentrations. It was found that 4 of the 22 screened proteins successfully upregulated IL-10 secretion when present at ˜100 nM concentrations (FIG. 4a).

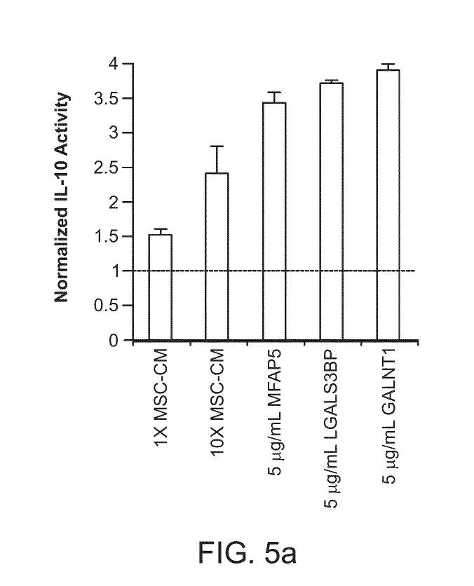

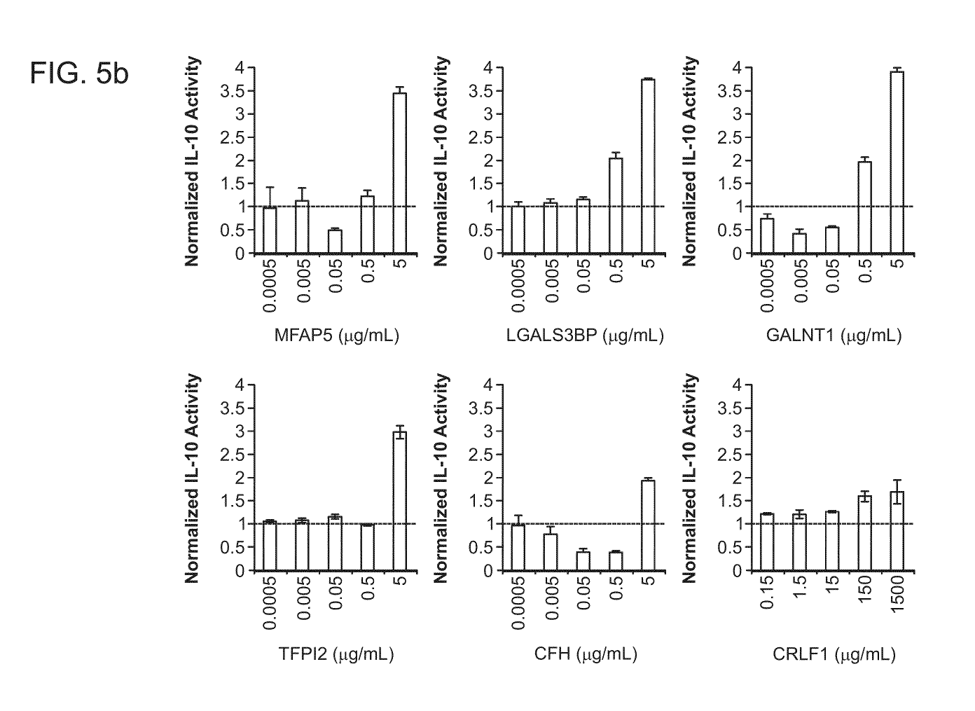

Recombinant molecules were obtained as products of the genes listed in Table 2, which represent the secreted molecule fraction of genes upregulated in LPS-pre-stimulated MSCs compared to MSCs and fibroblasts. The molecules were diluted in RPMI 1640 and activity was evaluated using the IL-10 assay (FIG. 5 and FIG. 4a). The four most potent molecules were the protein products of the genes LGALS3BP, MFAP5, GALNT1 and PENK, and all induced an increase of IL-10 production superior to that induced by 10×MSC-CM. FIG. 5 shows the increased production of IL-10 in leukocytes incubated with three of these molecules and reveals a dose-dependency over several orders of magnitude of dilution in RPMI 1640.

To confirm that BMSC-CM contained the four proteins, polypeptide N-acetylgalactosaminyltransferase 1 (GALNT1), galectin-3-binding protein (LGALS3BP), MFAP5 and PENK, we performed Western blots for GALNT1, MFAP5 and PENK, and used an ELISA for LGALS3BP (FIG. 4b). Clear bands were observed for MFAP5 and ELISA results showed LGALS3BP to be present at ng/mL concentrations in the BMSC-CM. GALNT1 and PENK were not present at detectable levels, even when the CM was concentrated 100-fold. We also performed proteomic LC/MS on bulk BMSC-CM and identified several of the 22 proteins in our enriched library, including LGALS3BP (FIG. 4c). Nevertheless, LC/MS failed to discern 18 of the 22 proteins (81%) from the enriched library, including GALNT1, MFAP5 and PENK. Taken together, these results demonstrate an unprecedented hit rate of protein discovery via EPS (18%), which is several orders of magnitude higher than traditional high throughput approaches that achieve in vitro rates on the order of 0.03-0.2% (Mayer, T. et al. Small molecule inhibitor of mitotic spindle bipolarity identified in a phenotype-based screen. Science 286, 971 (1999); Kwok, T. et al. A small-molecule screen in C. elegans yields a new calcium channel antagonist. Nature 441, 91-95 (2006); and Hung, D., Shakhnovich, E., Pierson, E. & Mekalanos, J. Small-molecule inhibitor of Vibrio cholerae virulence and intestinal colonization. Science 310, 670 (2005); all of which are incorporated herein by reference).

### Example 3

### Treatment of Colitic Mice with Factors Identified in the Differential Gene Expression Analysis

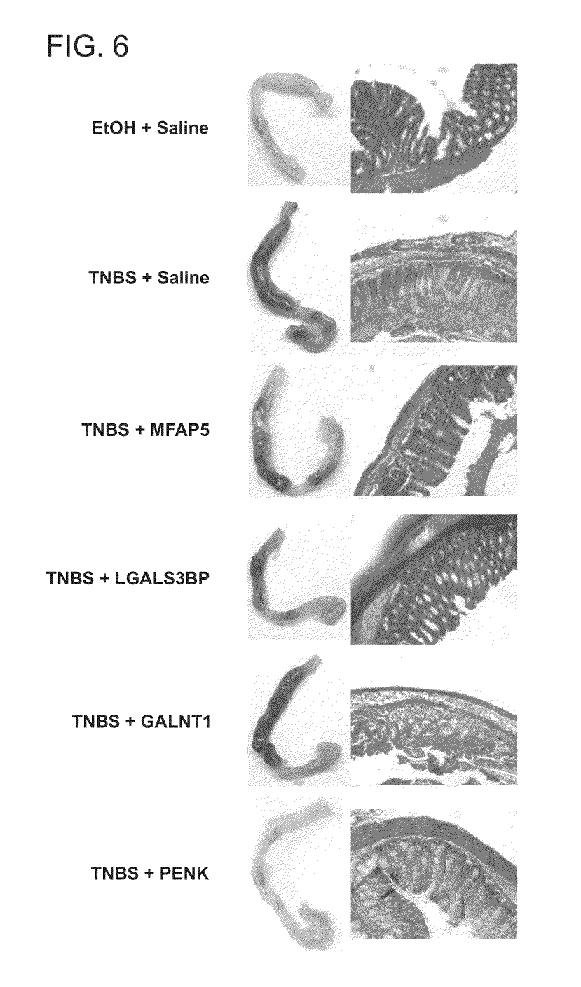

To test whether this observed effect was reproducible in vivo, animals were subjected to TNBS colitis followed by treatment with the four most potent molecules, LGALS3BP, MFAP5, GALNT1 and PENK. As seen in FIG. 6, MFAP5, LGALS3BP and PENK provided significant attenuation of colitis compared to controls. MFAP5, LGALS3BP and PENK all protected the colonic epithelium from the hallmark features of TNBS colitis: necrosis, inflammatory infiltrate and loss of crypts. Especially at the microscopic level, the tissue of the MFAP5, LGALS3BP and PENK treated animals looked mostly normal with minor foci of inflammation and edema.

### Example 4

### Suppression of Pro-Inflammatory Cytokines by MSC Factors and Active, Individual Agents Identified by Differential Gene Expression Analysis

Preparations and use of MSC-CM, Fb-CM, LPS-stimulated MSC-CM, leukocytes were as described in Examples 1-3 and the Figures. These conditioned media preparations were added to leukocyte cultures for 16 hours, after which the culture was stimulated with LPS at 10 μg/ml. The medium from this culture was collected 24 hours after LPS stimulation of leukocytes and analyzed using an IFN-γ ELISA.

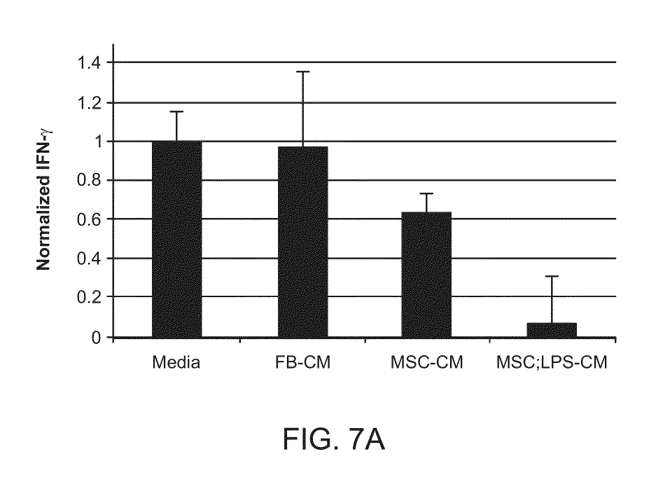

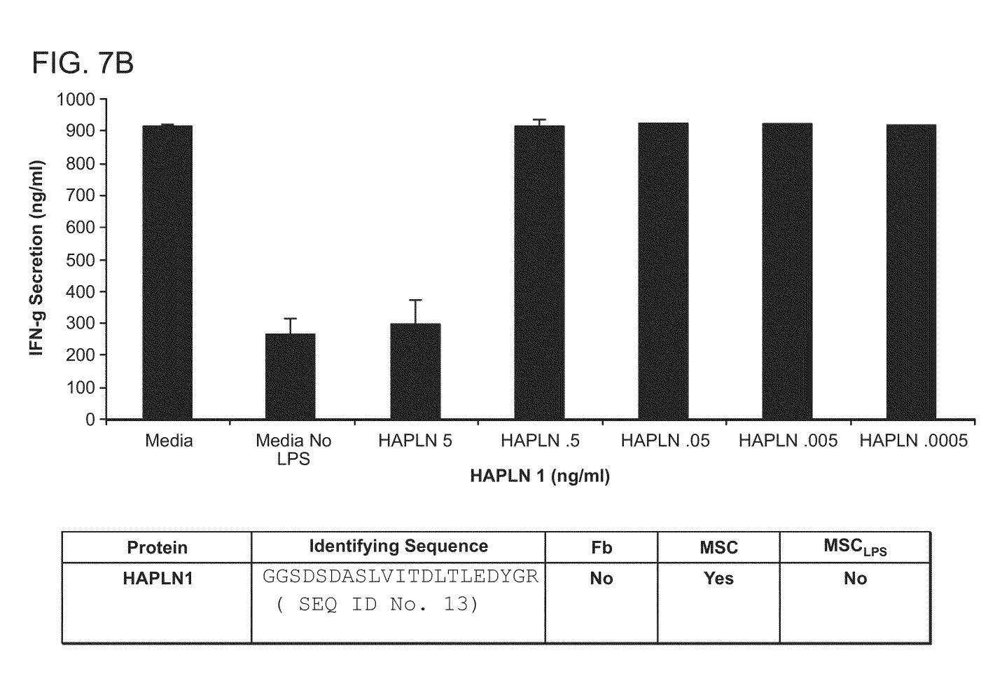

MSC-CM was found to cause a significant decrease in IFN-γ production of the cultured leukocytes once stimulated with LPS, as seen in FIG. 7. As also seen in this Figure, the IFN-γ production of the leukocytes incubated with Fb-CM was similar in amount to using the same volume of RPMI medium as a control. In contrast, LPS-stimulated MSC-CM reduced IFN-γ production even lower than MSCs that had not received prior stimulation with LPS. These results indicated that MSCs secrete specific factors that decrease IFN-γ production from leukocytes in a manner that fit the prerequisites for our differential gene expression analysis. From the analysis performed in Example 2, HAPLN1 was identified and observed to independently reduce IFN-γ production from leukocytes when used in a purified, recombinant form. These results indicated a dose-dependency of IFN-γ production over several orders of magnitude of diluted HAPLN1 in RPMI 1640. FIG. 7 also states the identifying sequence of HAPLN1 found in MSC-CM by size separation liquid chromatography followed by mass spectrometry.

### Example 5

### Treatment of LPS-Treated Mice with Factors Identified in the Differential Gene Expression Analysis

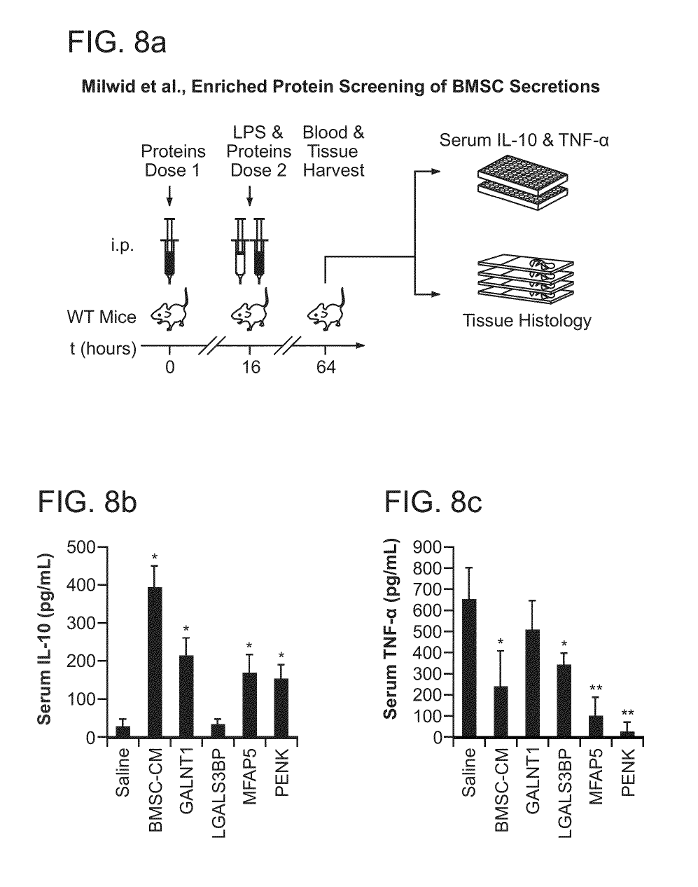

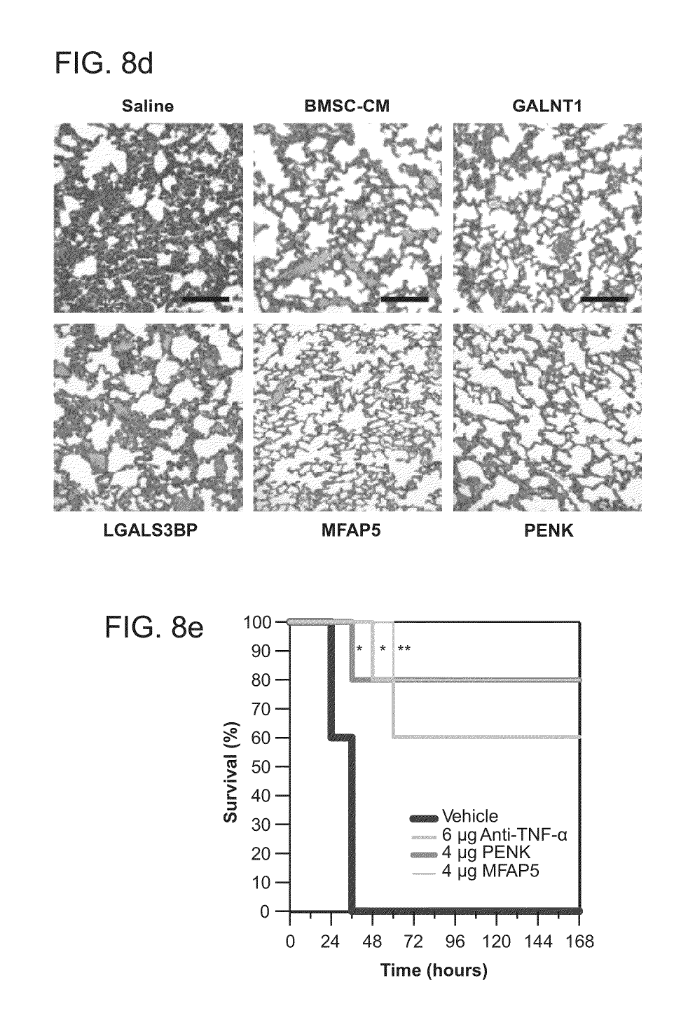

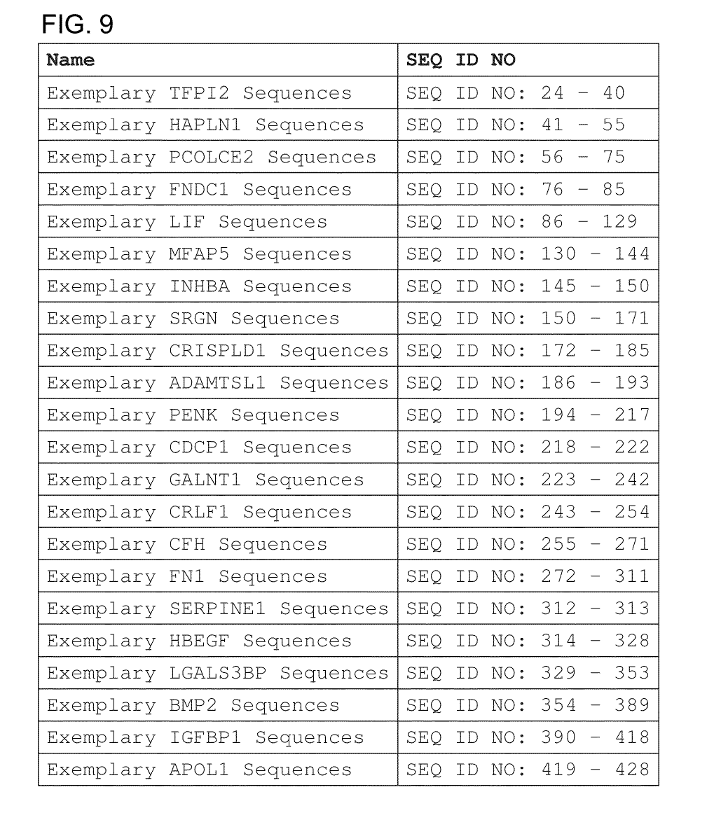

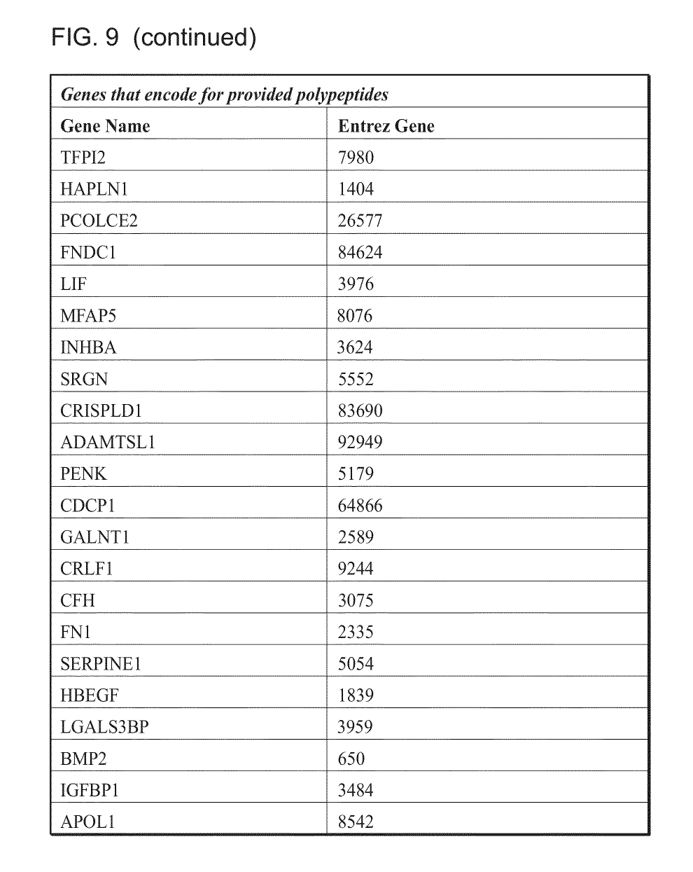

The four hits from the enriched recombinant protein screen were tested for activity in vivo in animals challenged with a sub-lethal dose of LPS (FIG. 8a). Significantly elevated serum IL-10 levels were observed in LPS-treated mice receiving BMSC-CM, GALNT1, MFAP5 and PENK compared to baseline vehicle control (saline), but no IL-10 response from LGALS3BP (FIG. 8b). Serum TNF-α was also significantly suppressed in mice receiving BMSC-CM, LGALS3BP, MFAP5 and PENK, but not GALNT1 (FIG. 8c). In addition, MFAP5 and PENK demonstrated superior TNF-α suppression compared to BMSC-CM. These results suggest that the different factors may influence different pathways, and the present invention encompasses the recognition that combinations of these factors can be used to treat multiple pathways in parallel.

The effects of cytokine modulation were apparent in the lung histology of the animals (FIG. 8d). In vehicle treated animals, edema, inflammatory infiltrate and alveolar collapse were evident in all lung fields. MFAP5 showed superior lung protection, preventing widespread inflammatory cell infiltrate and edema, thereby preserving the structure of the alveoli in the majority of the lungs. PENK, BMSC-CM and GALNT1 showed moderate protection with apparent inflammatory infiltrate, emphysematous changes and alveolar wall thickening, but no frank alveolar collapse.

The two most promising proteins, MFAP5 and PENK, were then tested in mice challenged with a lethal dose of LPS. Compared to anti-TNF-α, both proteins exhibited similar protection and provided a significant survival benefit.

### REFERENCES

Aggarwal, S, and M. Pittenger (2005). “Human mesenchymal stem cells modulate allogeneic immune cell responses.” Blood 105(4): 1815-1822.
Aggarwal, S, and M. F. Pittenger (2005). “Human mesenchymal stem cells modulate allogeneic immune cell responses.” Blood 105(4): 1815-22.
Albig, A., D. Becenti, et al. (2008). “Microfibril-associate glycoprotein-2 (MAGP-2) promotes angiogenic cell sprouting by blocking notch signaling in endothelial cells.” Microvascular research.
Amado, L., A. Saliaris, et al. (2005). “Cardiac repair with intramyocardial injection of allogeneic mesenchymal stem cells after myocardial infarction.” Proceedings of the National Academy of Sciences 102(32): 11474-11479.
Arnulf, B., S. Lecourt, et al. (2007). “Phenotypic and functional characterization of bone marrow mesenchymal stem cells derived from patients with multiple myeloma.” Leukemia 21(1): 158-63.
Augello, A., R. Tasso, et al. (2005). “Bone marrow mesenchymal progenitor cells inhibit lymphocyte proliferation by activation of the programmed death 1 pathway.” European Journal of Immunology 35(5).
Auvinen, P., R. Tammi, et al. (2000). “Hyaluronan in peritumoral stroma and malignant cells associates with breast cancer spreading and predicts survival.” American Journal of Pathology 156(2): 529.
Barta, E., F. Deak, et al. (1993). “Evolution of the hyaluronan-binding module of link protein.”Biochemical Journal 292(Pt 3): 947.
Bartholomew, A., C. Sturgeon, et al. (2002). “Mesenchymal stem cells suppress lymphocyte proliferation in vitro and prolong skin graft survival in vivo.” Experimental Hematology 30(1): 42-48.
Behar, O., H. Ovadia, et al. (1994). “Lipopolysaccharide induces proenkephalin gene expression in rat lymph nodes and adrenal glands.” Endocrinology 134(1): 475-81.
Beyth, S., Z. Borovsky, et al. (2005). “Human mesenchymal stem cells alter antigen-presenting cell maturation and induce T-cell unresponsiveness.” Blood 105(5): 2214-9.
Block, G., S. Ohkouchi, et al. (2009). “Multipotent stromal cells are activated to reduce apoptosis in part by upregulation and secretion of stanniocalcin-1.” Stem Cells 27(3).
Blundell, C., D. Mahoney, et al. (2003). “The link module from ovulation- and inflammation-associated protein TSG-6 changes conformation on hyaluronan binding.” Journal of Biological Chemistry 278(49): 49261.
Brazowski, E., I. Dotan, et al. (2009). “Galectin-3 expression in pouchitis in patients with ulcerative colitis who underwent ileal pouch-anal anastomosis (IPAA).” Pathol Res Pract 205(8): 551-8.
Breton, C., R. Oriol, et al. (1996). “Sequence alignment and fold recognition of fucosyltransferases.” Glycobiology 6(7).
Corcione, A., F. Benvenuto, et al. (2006). “Human mesenchymal stem cells modulate B-cell functions.” Blood 107(1): 367-372.
D'Ostilio, N., G. Sabatino, et al. (1996). “90K (Mac-2 BP) in human milk.” Clin Exp Immunol 104(3): 543-6.
Di Nicola, M., C. Carlo-Stella, et al. (2002). “Human bone marrow stromal cells suppress T-lymphocyte proliferation induced by cellular or nonspecific mitogenic stimuli.” Blood 99(10): 3838.
Djouad, F., P. Plence, et al. (2003). “Immunosuppressive effect of mesenchymal stem cells favors tumor growth in allogeneic animals.” Blood 102(10): 3837-44.
Djouad, F., P. Plence, et al. (2003). “Immunosuppressive effect of mesenchymal stem cells favors tumor growth in allogeneic animals.” Blood 102(10): 3837-3844.
Fiorina, P., M. Jurewicz, et al. (2009). “Immunomodulatory Function of Bone Marrow-Derived Mesenchymal Stem Cells in Experimental Autoimmune Type 1 Diabetes.” The Journal of Immunology 183(2): 993.
Gerdoni, E., B. Gallo, et al. (2007). “Mesenchymal stem cells effectively modulate pathogenic immune response in experimental autoimmune encephalomyelitis.” Annals of Neurology 61(3): 219.
Gibson, M., G. Hatzinikolas, et al. (1996). “Further characterization of proteins associated with elastic fiber microfibrils including the molecular cloning of MAGP-2 (MP25).” Journal of Biological Chemistry 271(2): 1096.
Gibson, M., D. Leavesley, et al. (1999). “Microfibril-associated Glycoprotein-2 Specifically Interacts with a Range of Bovine and Human Cell Types via alpha Vbeta 3 Integrin.” Journal of Biological Chemistry 274(19): 13060.
Goumon, Y., K. Lugardon, et al. (1998). “Characterization of antibacterial COOH-terminal proenkephalin-A-derived peptides (PEAP) in infectious fluids. Importance of enkelytin, the antibacterial PEAP209-237 secreted by stimulated chromaffin cells.” J Biol Chem 273(45): 29847-56.
Hook, V. Y., S, Noctor, et al. (1999). “Evidence for functional localization of the proenkephalin-processing enzyme, prohormone thiol protease, to secretory vesicles of chromaffin cells.” Endocrinology 140(8): 3744-54.
Horwitz, E., D. Prockop, et al. (1999). “Transplantability and therapeutic effects of bone marrow-derived mesenchymal cells in children with osteogenesis imperfecta.” Nature Medicine 5(3): 309-313.
Imberty, A., V. Piller, et al. (1997). “Fold recognition and molecular modeling of a lectin-like domain in UDP-GalNAc: polypeptide N-acetylgalactosaminyltransferases.” Protein Engineering Design and Selection 10(12): 1353.
Ivanova, A., C. Goparaju, et al. (2009). “Protumorigenic Role of HAPLN1 and Its IgV Domain in Malignant Pleural Mesothelioma.” Clinical Cancer Research 15(8): 2602.
Jiang, X. X., Y. Zhang, et al. (2005). “Human mesenchymal stem cells inhibit differentiation and function of monocyte-derived dendritic cells.” Blood 105(10): 4120-6.
Klyushnenkova, E., J. Mosca, et al. (1998). “Human mesenchymal stem cells suppress allogeneic T cell responses in vitro: implications for allogeneic transplantation.” Blood 92(suppl 1): 2652.
Klyushnenkova, E., J. D. Mosca, et al. (2005). “T cell responses to allogeneic human mesenchymal stem cells: immunogenicity, tolerance, and suppression.” J Biomed Sci 12(1): 47-57.
Koths, K., E. Taylor, et al. (1993). “Cloning and characterization of a human Mac-2-binding protein, a new member of the superfamily defined by the macrophage scavenger receptor cysteine-rich domain.” J Biol Chem 268(19): 14245-9.
Lazarus, H., S. Haynesworth, et al. (1995). “Ex vivo expansion and subsequent infusion of human bone marrow-derived stromal progenitor cells (mesenchymal progenitor cells): implications for therapeutic use.” Bone Marrow Transplantation 16(4): 557.
Le Blanc, K. (2003). “Immunomodulatory effects of fetal and adult mesenchymal stem cells.”Cytotherapy 5(6): 485-9.
Le Blanc, K., I. Rasmusson, et al. (2004). “Treatment of severe acute graft-versus-host disease with third party haploidentical mesenchymal stem cells.” Lancet 363(9419): 1439-41.
Le Blanc, K., L. Tammik, et al. (2003). “Mesenchymal stem cells inhibit and stimulate mixed lymphocyte cultures and mitogenic responses independently of the major histocompatibility complex.” Scandinavian Journal of Immunology 57(1): 11.
Lee, R., A. Pulin, et al. (2009). “Intravenous hMSCs Improve Myocardial Infarction in Mice because Cells Embolized in Lung Are Activated to Secrete the Anti-inflammatory Protein TSG-6.” Cell Stem Cell 5(1): 54-63.
Li, Y. P., S. Paczesny, et al. (2008). “Human Mesenchymal Stem Cells License Adult CD34+ Hemopoietic Progenitor Cells to Differentiate into Regulatory Dendritic Cells through Activation of the Notch Pathway.” J Immunol 180(3): 1598-608.
Liechty, K., T. MacKenzie, et al. (2000). “Human mesenchymal stem cells engraft and demonstrate site-specific differentiation after in utero transplantation in sheep.” Nature Medicine 6(11): 1282-1286.
Liu, J., X. F. Lu, et al. (2004). “Suppression of human peripheral blood lymphocyte proliferation by immortalized mesenchymal stem cells derived from bone marrow of Banna Minipig inbred-line.” Transplant Proc 36(10): 3272-5.
Marchetti, A., N. Tinari, et al. (2002). “Expression of 90K (Mac-2 BP) correlates with distant metastasis and predicts survival in stage I non-small cell lung cancer patients.” Cancer Res 62(9): 2535-9.
Meisel, R., A. Zibert, et al. (2004). “Human bone marrow stromal cells inhibit allogeneic T-cell responses by indoleamine 2,3-dioxygenase-mediated tryptophan degradation.” Journal of the American Society of Nephrology 103(12): 4619-4621.
Metz-Boutigue, M. H., A. E. Kieffer, et al. (2003). “Innate immunity: involvement of new neuropeptides.” Trends Microbiol 11(12): 585-92.
Miyahara, Y., N. Nagaya, et al. (2006). “Monolayered mesenchymal stem cells repair scarred myocardium after myocardial infarction.” Nature Medicine 12(4): 459-465.
Miyamoto, A., R. Lau, et al. (2006). “Microfibrillar proteins MAGP-1 and MAGP-2 induce Notch1 extracellular domain dissociation and receptor activation.” Journal of Biological Chemistry 281(15): 10089.
Neame, P., J. Christner, et al. (1986). “The primary structure of link protein from rat chondrosarcoma proteoglycan aggregate.” Journal of Biological Chemistry 261(8): 3519.
Németh, K., A. Leelahavanichkul, et al. (2008). “Bone marrow stromal cells attenuate sepsis via prostaglandin E2-dependent reprogramming of host macrophages to increase their interleukin-10 production.” Nature Medicine 15: 42-49.
Ortiz, L., F. Gambelli, et al. (2003). “Mesenchymal stem cell engraftment in lung is enhanced in response to bleomycin exposure and ameliorates its fibrotic effects.” Proceedings of the National Academy of Sciences 100(14): 8407-8411.
Penner, A., M. Rock, et al. (2002). “Microfibril-associated glycoprotein-2 interacts with fibrillin-1 and fibrillin-2 suggesting a role for MAGP-2 in elastic fiber assembly.” Journal of Biological Chemistry 277(38): 35044-35049.
Pittenger, M. (2009). “Sleuthing the Source of Regeneration by MSCs.” Cell Stem Cell 5(1): 8-10.
Poggi, A., C. Prevosto, et al. (2005). “Interaction between Human NK Cells and Bone Marrow Stromal Cells Induces NK Cell Triggering: Role of NKp30 and NKG2D Receptors 1.” The Journal of Immunology 175(10): 6352-6360.
Potian, J. A., H. Aviv, et al. (2003). “Veto-like activity of mesenchymal stem cells: functional discrimination between cellular responses to alloantigens and recall antigens.” J Immunol 171(7): 3426-34.
Prevosto, C., M. Zancolli, et al. (2007). “Generation of CD4+ or CD8+ regulatory T cells upon mesenchymal stem cell-lymphocyte interaction.” Haematologica 92(7): 881-8.
Raffaghello, L., G. Bianchi, et al. (2008). “Human mesenchymal stem cells inhibit neutrophil apoptosis: a model for neutrophil preservation in the bone marrow niche.” Stem Cells 26(1): 151.
Rasmusson, I., K. Le Blanc, et al. (2007). “Mesenchymal stem cells stimulate antibody secretion in human B cells.” Scand J Immunol 65(4): 336-43.
Rasmusson, I., M. Uhlin, et al. (2007). “Mesenchymal stem cells fail to trigger effector functions of cytotoxic T lymphocytes.” J Leukoc Biol 82(4): 887-93.
Resnick, D., A. Pearson, et al. (1994). “The SRCR superfamily: a family reminiscent of the Ig superfamily.” Trends Biochem Sci 19(1): 5-8.
Rojas, M., J. Xu, et al. (2005). “Bone marrow derived mesenchymal stem cells in repair of the injured lung.” American Journal of Respiratory Cell and Molecular Biology: 2004.
Rosenberg, I., B. J. Cherayil, et al. (1991). “Mac-2-binding glycoproteins. Putative ligands for a cytosolic beta-galactoside lectin.” J Biol Chem 266(28): 18731-6.
Salzet, M. and A. Tasiemski (2001). “Involvement of pro-enkephalin-derived peptides in immunity.” Dev Comp Immunol 25(3): 177-85.
Sasaki, T., C. Brakebusch, et al. (1998). “Mac-2 binding protein is a cell-adhesive protein of the extracellular matrix which self-assembles into ring-like structures and binds beta1 integrins, collagens and fibronectin.” EMBO J 17(6): 1606-13.
Shake, J., P. Gruber, et al. (2002). “Mesenchymal stem cell implantation in a swine myocardial infarct model: engraftment and functional effects.” The Annals of Thoracic Surgery 73(6): 1919-1926.
Sharp, B. M., S. Roy, et al. (1998). “Evidence for opioid receptors on cells involved in host defense and the immune system.” J Neuroimmunol 83(1-2): 45-56.
Sotiropoulou, P., S. Perez, et al. (2006). “Interactions between human mesenchymal stem cells and natural killer cells.” Stem Cells 24(1): 74-85.
Spaggiari, G., A. Capobianco, et al. (2008). “Mesenchymal stem cells inhibit natural killer-cell proliferation, cytotoxicity, and cytokine production: role of indoleamine 2,3-dioxygenase and prostaglandin E2.” Blood 111(3): 1327.
Spaggiari, G., A. Capobianco, et al. (2006). “Mesenchymal stem cell-natural killer cell interactions: evidence that activated NK cells are capable of killing MSCs, whereas MSCs can inhibit IL-2-induced NK-cell proliferation.” Blood 107(4): 1484-1490.
Spicer, A., A. Joo, et al. (2003). “A Hyaluronan Binding Link Protein Gene Family Whose Members Are Physically Linked Adjacent to Chrondroitin Sulfate Proteoglycan Core Protein Genes.” Journal of Biological Chemistry 278(23): 21083.
Tenno, M., S. Toba, et al. (2002). “Identification of two cysteine residues involved in the binding of UDP-GalNAc to UDP-GalNAc: polypeptide N-acetylgalactosaminyltransferase 1 (GalNAc-T1).” European Journal of Biochemistry 269(17): 4308-4316.
Togel, F., Z. Hu, et al. (2005). “Administered mesenchymal stem cells protect against ischemic acute renal failure through differentiation-independent mechanisms.” American Journal of Physiology Renal Physiology 289(1): F31.
Togel, F., K. Weiss, et al. (2007). “Vasculotropic, paracrine actions of infused mesenchymal stem cells are important to the recovery from acute kidney injury.” American Journal of Physiology Renal Physiology 292(5): F1626.
Torlakovic, E. E., V. Keeler, et al. (2009). “Cyclophilin C-associated protein (CyCAP) knock-out mice spontaneously develop colonic mucosal hyperplasia and exaggerated tumorigenesis after treatment with carcinogen azoxymethane.” BMC Cancer 9: 251.
Traggiai, E., S. Volpi, et al. (2008). “Bone marrow-derived mesenchymal stem cells induce both polyclonal expansion and differentiation of B cells isolated from healthy donors and systemic lupus erythematosus patients.” Stem Cells 26(2): 562-9.
Trahey, M. and I. L. Weissman (1999). “Cyclophilin C-associated protein: a normal secreted glycoprotein that down-modulates endotoxin and proinflammatory responses in vivo.” Proc Natl Acad Sci USA 96(6): 3006-11.
Tse, W., W. Beyer, et al. (2000). “Bone marrow derived mesenchymal stem cells suppress T cell activation without inducing allogeneic anergy.” Blood 96: 241a.
Uccelli, A., L. Moretta, et al. (2008). “Mesenchymal stem cells in health and disease.” Nature Reviews Immunology 8(9): 726-736.
Ullrich, A., I. Sures, et al. (1994). “The secreted tumor-associated antigen 90K is a potent immune stimulator.” J Biol Chem 269(28): 18401-7.
van Poll, D., B. Parekkadan, et al. (2008). “Mesenchymal Stem Cell Therapy for Protection and Repair of Injured Vital Organs.” Cellular and Molecular Bioengineering 1(1): 42-50.
Weir, C., A. McNeill, et al. (2006). “Critical role of preproenkephalin in experimental autoimmune encephalomyelitis.” J Neuroimmunol 179(1-2): 18-25.
White, T., E. Bennett, et al. (1995). “Purification and cDNA cloning of a human UDP-N-acetyl-alpha-D-galactosamine: polypeptide N-acetylgalactosaminyltransferase.” Journal of Biological Chemistry 270(41): 24156.
Yagi, H., B. Parekkadan, et al. (2009). “Long-Term Superior Performance of a Stem Cell/Hepatocyte Device for the Treatment of Acute Liver Failure.” Tissue Engineering Part A.
Zhang, W., W. Ge, et al. (2004). “Effects of mesenchymal stem cells on differentiation, maturation, and function of human monocyte-derived dendritic cells.” Stem Cells Dev 13(3): 263-71.
Zhu, G., M. Allende, et al. (1998). “Two soluble glycosyltransferases glycosylate less efficiently in vivo than their membrane bound counterparts.” Glycobiology 8(8): 831.

### EQUIVALENTS

The foregoing has been a description of certain non-limiting embodiments in accordance with the invention. Those skilled in the art will recognize, or be able to ascertain using no more than routine experimentation, many equivalents to the specific embodiments described herein. Those of ordinary skill in the art will appreciate that various changes and modifications to this description may be made without departing from the spirit or scope of the present invention, as defined in the following claims.

All literature and similar material cited in this application, including, but not limited to, patents, patent applications, articles, books, treatises, and web pages, regardless of the format of such literature and similar materials, are expressly incorporated by reference in their entirety.

It is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It should be understood that, in general, where the invention, or aspects of the invention, is/are referred to as comprising particular elements, features, steps, etc., certain embodiments or aspects consist, or consist essentially of, such elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments, to the tenth of the unit of the lower limit of the range. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment may be explicitly excluded from any one or more of the claims. Any embodiment, element, feature, application, or aspect of the compositions and/or methods (e.g., any marrow stromal cell [MSC] polypeptide, any characteristic sequence element of an MSC polypeptide, any method of manufacturing MSC polypeptides, any route or location of administration of MSC polypeptides and/or compositions thereof, any purpose for which a composition comprising MSC polypeptides is administered, etc.), can be excluded from any one or more claims. For purposes of brevity, all of the embodiments in which one or more elements, features, purposes, or aspects are excluded are not set forth explicitly herein.

1. A pharmaceutical composition comprising:
an active agent comprising a polypeptide selected from the group consisting of GALNT1 polypeptides, LGALS3BP polypeptides, MFAP5 polypeptides, HAPLN1 polypeptides, PENK polypeptides, and combinations thereof; and
a pharmaceutically acceptable carrier or excipient;
the composition being characterized in that:
the polypeptide is present in an amount sufficient to cause a change selected from the group consisting of:
an increase in production of at least one anti-inflammatory agent by leukocytes in culture as compared with a level observed under otherwise comparable conditions absent the polypeptide;
a decrease in production of at least one pro-inflammatory agent by leukocytes in culture as compared with a level observed under otherwise comparable conditions absent the polypeptide; and
combinations thereof.

2. The pharmaceutical composition of claim 1, wherein the polypeptide comprises one or more GALNT1 polypeptide.

3. The pharmaceutical composition of claim 1, wherein the polypeptide comprises one or more LGALS3BP polypeptide.

4. The pharmaceutical composition of claim 1, wherein polypeptide comprises one or more MFAP5 polypeptide.

5. The pharmaceutical composition of claim 1, wherein the polypeptide comprises one or more PENK polypeptide.

6. The pharmaceutical composition of claim 1, wherein the polypeptide comprises one or more HAPLN1 polypeptide.

7. The pharmaceutical composition of claim 2, wherein the GALNT1 polypeptide is present at a concentration within one order of magnitude of that at which a polypeptide selected from the group consisting of SEQ ID NOs: 223-242 is found in human serum.

8. The pharmaceutical composition of claim 3, wherein the LGALS3BP polypeptide is present at a concentration within one order of magnitude of that at which a polypeptide selected from the group consisting of SEQ ID NOs: 329-353 is found in human serum.

9. The pharmaceutical composition of claim 4, wherein the MFAP5 polypeptide is present at a concentration within one order of magnitude of that at which a polypeptide selected from the group consisting of SEQ ID NOs: 130-144 is found in human serum.

10. The pharmaceutical composition of claim 5, wherein the PENK polypeptide is present at a concentration within one order of magnitude of that at which a polypeptide selected from the group consisting of SEQ ID NOs: 194-217 is found in human serum.

11. The pharmaceutical composition of claim 6, wherein the HAPLN1 polypeptide is present at a concentration within one order of magnitude of that at which a polypeptide selected from the group consisting of SEQ ID NOs: 41-55 is found in human serum.

12-16. (canceled)

17. The pharmaceutical composition of claim 1, wherein the polypeptide comprises at least two distinct polypeptides selected from the group consisting of GALNT1 polypeptides, LGALS3BP polypeptides, MFAP5 polypeptides, HAPLN1 polypeptides, PENK polypeptides, and combinations thereof.

18. The pharmaceutical composition of claim 17, wherein the polypeptide comprises at least one LGALS3BP polypeptide, and at least one MFAP5 polypeptide.

19. The pharmaceutical composition of claim 17, wherein the polypeptide comprises at least one LGALS3BP polypeptide, and at least one PENK polypeptide.

20. The pharmaceutical composition of claim 17, wherein the polypeptide comprises at least one LGALS3BP polypeptide, and at least one HAPLN1 polypeptide.

21. The pharmaceutical composition of claim 17, wherein the polypeptide comprises at least one MFAP5 polypeptide, and at least one PENK polypeptide.

22. The pharmaceutical composition of claim 17, wherein the polypeptide comprises at least one MFAP5 polypeptide, and at least one HAPLN1 polypeptide.

23. The pharmaceutical composition of claim 17, wherein the polypeptide comprises at least one PENK polypeptide, and at least one HAPLN1 polypeptide.

24. The pharmaceutical composition of claim 1, wherein the polypeptide comprises at least one distinct polypeptide selected from the group consisting of GALNT1 polypeptides, LGALS3BP polypeptides, MFAP5 polypeptides, HAPLN1 polypeptides, PENK polypeptides in combination with at least one distinct polypeptide from the group consisting of TFIP2 polypeptides, HB-EGF polypeptides, PCOLCE2 polypeptides, FNDC1 polypeptides, LIF polypeptides, INHBA polypeptides, SRGN polypeptides, CRISPLD1 polypeptides, ADAMTSL1 polypeptides, CDCP1 polypeptides, CRLF1 polypeptides, CFH polypeptides, FN1 polypeptides, SERPINE1 polypeptides, BMP2 polypeptides, IGFBP1 polypeptides, APOL1 polypeptides, and combinations thereof.

25-33. (canceled)

34. A pharmaceutical composition comprising:
an active agent comprising an antibody specific to a polypeptide selected from the group consisting of GALNT1 polypeptides, LGALS3BP polypeptides, MFAP5 polypeptides, HAPLN1 polypeptides, PENK polypeptides; and
a pharmaceutically acceptable carrier or excipient;
the composition being characterized in that:
the antibody is present in an amount sufficient to inhibit, as compared with a level observed under otherwise comparable conditions absent the antibody, an activity characteristic of the polypeptide to which the antibody is specific, the ability being selected from the group consisting of:
an ability to increase production of at least one anti-inflammatory agent by leukocytes in culture as compared with a level observed under otherwise comparable conditions absent the polypeptides;
an ability to decrease production of at least one pro-inflammatory agent by leukocytes in culture as compared with a level observed under otherwise comparable conditions absent the polypeptide; and
combinations thereof.

35. The pharmaceutical composition of claim 34, wherein the antibody is specific for one or more GALNT1 polypeptide.

36. The pharmaceutical composition of claim 34, wherein the antibody is specific for one or more LGALS3BP polypeptide.

37. The pharmaceutical composition of claim 34, wherein antibody is specific for one or more MFAP5 polypeptide.

38. The pharmaceutical composition of claim 34, wherein the antibody is specific for one or more PENK polypeptide.

39. The pharmaceutical composition of claim 34, wherein the antibody is specific for one or more HAPLN1 polypeptide.

40. The pharmaceutical composition of claim 35 wherein a GALNT1 polypeptide has an amino acid sequence that shows at least 50% overall identity with a polypeptide selected from the group consisting of SEQ ID NOs: 223-242.

41. The pharmaceutical composition of claim 36 wherein a LGALS3BP polypeptide has an amino acid sequence that shows at least 50% overall identity with a polypeptide selected from the group consisting of SEQ ID NOs: 329-353.

42. The pharmaceutical composition of claim 37 wherein a MFAP5 polypeptide has an amino acid sequence that shows at least 50% overall identity with a polypeptide selected from the group consisting of SEQ ID NOs: 130-144.

43. The pharmaceutical composition of claim 38 wherein a PENK polypeptide has an amino acid sequence that shows at least 50% overall identity with a polypeptide selected from the group consisting of SEQ ID NOs: 194-217.

44. The pharmaceutical composition of claim 39 wherein a HAPLN1 polypeptide has an amino acid sequence that shows at least 50% overall identity with a polypeptide selected from the group consisting of SEQ ID NOs: 41-55.

45-55. (canceled)

56. A method comprising steps of:
determining a level of a polypeptide selected from the group consisting of GALNT1 polypeptides, LGALS3BP polypeptides, MFAP5 polypeptides, HAPLN1 polypeptides, PENK polypeptides, and combinations thereof present in a sample;
based on the determined level, establishing likelihood that the sample is expected to have therapeutic activity.

57. The method of claim 56, wherein:
the step of determining comprises determining a level of a polypeptide selected from the group consisting of GALNT1 polypeptides, LGALS3BP polypeptides, MFAP5 polypeptides, HAPLN1 polypeptides, PENK polypeptides, and combinations thereof present in a sample that comprises one or more marrow stromal cells in a test sample; and
the step of establishing comprises likelihood that the sample contains marrow stromal cells expected to have therapeutic activity.
